(19) United States
(12) Patent Application Publication
(10) Pub. No.: US 2026/0000665 A1
(43) Pub. Date: Jan. 1, 2026

(54) ENANTIOMER OF AZOPODOPHYLLOTOXIN DERIVATIVE SU056

(71) Applicant: Oregon Health & Science University, Portland, OR (US)

(72) Inventors: Sanjay Malhotra, Portland, OR (US); Dhanir Tailor, Portland, OR (US); Arpit Dheeraj, Portland, OR (US)

(73) Assignee: Oregon Health & Science University, Portland, OR (US)

(21) Appl. No.: 18/992,473

(22) PCT Filed: Jul. 11, 2023
(86) PCT No.: PCT/US23/69965

§ 371 Date: Jan. 8, 2025

Related U.S. Application Data
(60) Provisional application No. 63/388,537, filed on Jul. 12, 2022.

(51) Int. Cl. A61K 31/4741; A61K 31/337; A61K 31/357; A61K 31/427; A61K 31/475; A61K 31/513; A61K 31/555; A61K 31/675; A61K 31/704; A61K 31/7068; A61K 33/243; A61P 35/00

(52) U.S. Cl. CPC A61K 31/4741; A61K 31/337; A61K 31/357; A61K 31/427; A61K 31/475; A61K 31/513; A61K 31/555; A61K 31/675; A61K 31/704; A61K 31/7068; A61K 33/243; A61P 35/00

(57) ABSTRACT

The present invention concerns a novel enantiomer of 9-(3-fluorophenyl)-5-(2-hydroxyethyl)-6,9-dihydro-[1,3]dioxolo[4,5-g]furo[3,4-b]quinolin-8 (5H)-one (SU056), as well as pharmaceutically acceptable salts thereof, pharmaceutical compositions comprising it, and its uses in medical treatments, particularly including cancer treatments.

### CROSS-REFERENCE TO RELATED APPLICATIONS

This is the 371 National Phase of International Application No. PCT/US23/69965, filed Jul. 11, 2023, which claims priority under 35 U.S.C. § 119 (e) to U.S. Provisional Patent Application No. 63/388,537, filed Jul. 12, 2022, which is hereby incorporated by reference in its entirety.

### FIELD OF THE INVENTION

The present invention concerns a novel levorotatory enantiomer of 9-(3-fluorophenyl)-5-(2-hydroxyethyl)-6,9-dihydro-[1,3]dioxolo[4,5-g]furo[3,4-b]quinolin-8(5H)-one (L-SU056) and its uses in medical treatments, particularly including cancer treatments.

### BACKGROUND OF THE INVENTION

Y box binding protein 1 (YB-1, YBX1) is a multifunctional cold shock protein that binds to DNA and RNA and has been associated with tumor progression and the emergence of treatment resistance (TR). It regulates DNA and RNA associated cellular events including mRNA transcription, splicing, packaging, stability, and translation (Lyabin et al., 2014, YB-1 protein: functions and regulation. Wiley Interdiscip Rev RNA, 5, 95-110). mRNA stabilization is an important event for sustained expression of any gene and YB-1 robustly stabilizes the mRNA via blocking the 5′ end from mRNA degradation (Evdokimova et al., 2001, The major mRNA-associated protein YB-1 is a potent 5′ cap-dependent mRNA stabilizer. EMBO J, 20, 5491-502). It was first described by Didier et al. as a negative regulator of the MHC class II molecule (Didier et al., 1988). The oncogenic role of YB-1 is well-characterized in many cancers and its amplified levels have been found in a large number of cancer cases (Goodarzi et al., Cell 161, 790-802, 2015). It increases the stability of short-lived mRNAs for multiple oncogenic proteins including c-myc (Laird-Offringa et al., 1990, Poly(A) tail shortening is the translation-dependent step in c-myc mRNA degradation. Mol Cell Biol, 10, 6132-40), c-fos (Blattner et al., 2000, UV-Induced stabilization of c-fos and other short-lived mRNAs. Mol Cell Biol, 20, 3616-25), cyclin B1 (Maity et al., 2011. Class III beta-tubulin (TUBB3): more than a biomarker in solid tumors? Curr Mol Med, 11, 726-31), HIF1α (Goodarzi et al., 2015, Endogenous tRNA-Derived Fragments Suppress Breast Cancer Progression via YBX1 Displacement. Cell, 161, 790-802), Snail (Evdokimova et al., 2009, Translational activation of snail1 and other developmentally regulated transcription factors by YB-1 promotes an epithelial-mesenchymal transition. Cancer Cell, 15, 402-15), and MDR1 (Bargou et al., 1997), which are associated with disease progression and treatment resistance. Genetic knockdown studies have demonstrated that inhibition of YB-1 significantly arrests proliferation and induces apoptosis in many cancer models, demonstrating its essential role in disease progression (Evdokimova et al., 2009, El-Naggar et al., 2015, Translational Activation of HIF1alpha by YB-1 Promotes Sarcoma Metastasis. Cancer Cell, 27, 682-97). YB-1 is associated with the development of treatment resistance (TR) via its role in activating proliferation, promoting cancer cell stemness, responding to growth factors, cytokines, cellular stress responses, and promoting drug efflux via the membrane P-glycoprotein ATP-dependent efflux pump ABCB1 (MDR1) (Bargou et al., 1997, Nuclear localization and increased levels of transcription factor YB-1 in primary human breast cancers are associated with intrinsic MDR1 gene expression. Nat Med, 3, 447-50; Saupe et al., 2015, Differential expression of the multidrug resistance 1 (MDR1) protein in prostate cancer cells is independent from anticancer drug treatment and Y box binding protein 1 (YB-1) activity. World J Urol, 33, 1481-6; and Mo et al., 2016, Human Helicase RECQL4 Drives Cisplatin Resistance in Gastric Cancer by Activating an AKT-YB1-MDR1 Signaling Pathway. Cancer Res, 76, 3057-66). It is also associated with alternative splicing of CD44 exon via binding to the A/C-rich region (Stickeler et al., 2001, The RNA binding protein YB-1 binds A/C-rich exon enhancers and stimulates splicing of the CD44 alternative exon v4. EMBO J, 20, 3821-30). The YB-1 gene is highly conserved and only ˜1% of cancer patients show the mutation, although it is nonetheless overexpressed in a wide range of cancers via alternative gene regulatory networks.

Ovarian cancer (OC) accounts for only 3% of all cancer cases in women, but nonetheless causes disproportionate mortality (Dietl, 2014, Revisiting the pathogenesis of ovarian cancer: the central role of the fallopian tube. Arch Gynecol Obstet, 289, 241-6; Jayson et al., 2014, Ovarian cancer. Lancet, 384, 1376-88; Agarwal and Kaye, 2003, Ovarian cancer: strategies for overcoming resistance to chemotherapy. Nat Rev Cancer, 3, 502-16). Surgical resection followed by chemotherapy is the main treatment strategy for OC patients. Platinum- and taxol-based drugs and their combination are the first-line treatment for the majority of OC patients (Seifter, 1997, Cancer: Principles and Practice of Oncology, 5th EditionVincent T. DeVita, Jr., Samuel Hellman, Steven A. Rosenberg, eds. Philadelphia: Lippincott-Raven Publishers, 1997.3125 pp, illus. ISBN 0-397-51573-4. JNCI: Journal of the National Cancer Institute, 89, 353-353). The majority of women are diagnosed with OC at Stage III+ and frequently develop TR and disease relapse. BRCA1/2 mutations, amplification of MYC, and upregulation MDR1 (ABCB1/P-gp) are the most common known causes of TR in OC (Zeng et al., 2018, Targeting MYC dependency in ovarian cancer through inhibition of CDK7 and CDK12/13. Elife, 7; Christie and Bowtell, 2017, Acquired chemotherapy resistance in ovarian cancer. Ann Oncol, 28, viii13-viii15; Sun et al., 2015, Integrative transcriptomics-based identification of cryptic drivers of taxol-resistance genes in ovarian carcinoma cells: Analysis of the androgen receptor. Oncotarget, 6, 27065-82). Patient-based studies have shown that MYC amplification is associated with disease progression and TR in many high-grade epithelial OC lesions (Jung et al., 2017, A Myc Activity Signature Predicts Poor Clinical Outcomes in Myc-Associated Cancers. Cancer Res, 77, 971-981; and Jung et al., 2018, Clinical Importance of Myc Family Oncogene Aberrations in Epithelial Ovarian Cancer. JNCI Cancer Spectrum, 2). Nuclear localization of YB-1 plays an important role in the regulation of MYC, MDR1, and CD44 (Kang et al., 2013, Role of focal adhesion kinase in regulating YB-1-mediated paclitaxel resistance in ovarian cancer. J Natl Cancer Inst, 105, 1485-95; Sobocan et al., 2020, The Communication Between the PI3K/AKT/mTOR Pathway and Y-box Binding Protein-1 in Gynecological Cancer. Cancers (Basel), 12). Analysis of high-grade ovarian serous carcinoma samples suggests that patients with higher YB-1 expression (median survival 48.5 months) had shorter term survival compared to lower YB-1 expression (median survival 65 months) (Kang et al., 2013). Primary surgical and chemotherapy treatments for OC may be followed by maintenance therapy, which includes prolonged drug usage such as paclitaxel and PARP inhibitors (olaparib, pazopanib, and niraparib), but unfortunately still with limited outcomes (Franzese et al., 2019, PARP inhibitors in ovarian cancer. Cancer Treat Rev, 73, 1-9). Literature from the last three decades strongly suggests that YB-1 could be a potential target to treat ovarian and other cancers including those in which treatment resistance has developed. Even after an extensive investigation, there were no significant efforts had been made to develop small molecule inhibitors that can directly inhibit the YB-1. Y-box-binding protein 1 (YB-1), encoded by the YBX1 gene, has been noted as modulating or regulating cellular signaling pathways and may be seen as a molecular marker for cancer progression and as a target for cancer therapies.

Lasham et al. describe in their review article YB-1: oncoprotein, prognostic marker and therapeutic target?, Biochem. J. (2013) 449, 11-13, how “YB-1 regulates multiple proliferation pathways, overrides cell-cycle check points, promotes replicative immortality and genomic instability, may regulate angiogenesis, has a role in invasion and metastasis, and promotes inflammation.” They further describe cell lines in which YB-1 reduction induced apoptosis or inhibited cell proliferation, including melanoma, fibrosarcoma, liver cancer, lung cancer, bladder cancer, multiple myeloma, paediatric glioblastoma, breast cancer (ER-negative), breast cancer (ER-positive), prostate cancer, and colon cancer cell lines.

Sobočan et al. (Cancers, 2020, 12, 205) describe dual targeting of Y-box-protein 1 (YB-1) and mTOR as improving the inhibition of carcinogenic activity in gynecological cancers, including ovarian, endometrial, fallopian tube, and cervical cancers.

The article Oncogenic Y-box binding protein-1 as an effective therapeutic target in drug-resistant cancer, Kuwano et al., Cancer Science, 2019, 110:1536-1543, describes the function of YBX2 in promoting transcriptional activation of the ABCB1 transporter gene, which has been associated as a transcriptional mechanism of how tumor multidrug resistance is acquired during chemotherapeutic treatments in human malignancies, including breast, lung, ovarian, prostate, colorectal, and gastric cancers.

The relationship between increased expression of YBX1 and melanoma is discussed in the article The increased expression of Y box-binding protein 1 in melanoma stimulates proliferation and tumor invasion, antagonizes apoptosis and enhances chemoresistance, Schittek et al., Int. J. Cancer: 120, 2110-2118 (2007). YB1 overexpression has also been associated with radio-resistance in colorectal cancer cells, as discussed by Kim et al., Mol. Cancer Ther., 30 Oct. 2019, 19 (2), 479-89.

WO 2019/178091 A1 (Malholtra et al., The Board of Trustees of the Leland Stanford Junior University) teaches novel N-hydroxyethyl didehydroazapodophyllotoxins as GBP1 inhibitors and methods for their use in overcoming treatment resistance in cancers. Included in the disclosure is the compound 9-(3-fluorophenyl)-5-(2-hydroxyethyl)-6,9-dihydro-[1,3]dioxolo[4,5-g]furo[3,4-b]quinolin-8(5H)-one (SU056).

There remains a need for small molecule inhibitors of YB-1 for pharmaceutical use, particularly those useful in treating platinum-resistant cancers.

### SUMMARY OF THE INVENTION

Provided is a composition comprising(S)-9-(3-fluorophenyl)-5-(2-hydroxyethyl)-6,9-dihydro-[1,3]dioxolo[4,5-g]furo[3,4-b]quinolin-8(5H)-one, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, wherein the composition is substantially free of (R)-9-(3-fluorophenyl)-5-(2-hydroxyethyl)-6,9-dihydro-[1,3]dioxolo[4,5-g]furo[3,4-b]quinolin-8(5H)-one, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

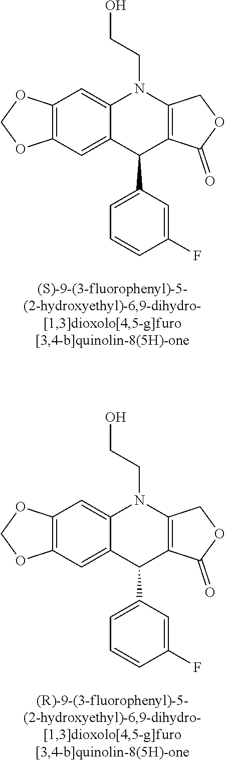 [SMILES: O=C1OCC2=C1[C@@H](c1cccc(F)c1)c1cc3c(cc1N2CCO)OCO3 ; O=C1OCC2=C1[C@H](c1cccc(F)c1)c1cc3c(cc1N2CCO)OCO3]

### BRIEF DESCRIPTION OF THE DRAWINGS

FIG. 1A presents a line graph of the results of a MTT cell viability assay in triple negative breast cancer cell line MDA-MB-231.

FIG. 1B presents a line graph of the results of a MTT cell viability assay in triple negative breast cancer cell line MDA-MB-468.

FIG. 1C presents a line graph of the results of a MTT cell viability assay in triple negative breast cancer cell line SUM159.

FIG. 1D presents a line graph of the results of a MTT cell viability assay in triple negative breast cancer cell line 4T1.

FIG. 2A presents a line graph of the results of a tumor xenograft study of SU056 and its enantiomers in 4T1 tumor xenograft in BALB/c mice plotted in tumor volume as a function of time.

FIG. 2B presents a line graph of the results of a tumor xenograft study of SU056 and its enantiomers in 4T1 tumor xenograft in BALB/c mice plotted in body weight as a function of time.

### DETAILED DESCRIPTION OF THE INVENTION

Provided is a composition comprising(S)-9-(3-fluorophenyl)-5-(2-hydroxyethyl)-6,9-dihydro-[1,3]dioxolo[4,5-g]furo[3,4-b]quinolin-8(5H)-one ((S)-SU056), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, in an enantiomeric excess to (R)-9-(3-fluorophenyl)-5-(2-hydroxyethyl)-6,9-dihydro-[1,3]dioxolo[4,5-g]furo[3,4-b]quinolin-8(5H)-one ((R)-SU056SU056), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

The compositions herein may also be referred to as composition comprising (L)-9-(3-fluorophenyl)-5-(2-hydroxyethyl)-6,9-dihydro-[1,3]dioxolo[4,5-g]furo[3,4-b]quinolin-8(5H)-one ((L)-SU056), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, in an enantiomeric excess to (D)-9-(3-fluorophenyl)-5-(2-hydroxyethyl)-6,9-dihydro-[1,3]dioxolo[4,5-g]furo[3,4-b]quinolin-8(5H)-one ((D)-SU056), or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

For the sake of brevity, reference to a pharmaceutically acceptable salt of (S)-SU056 or a pharmaceutically acceptable salt of (R)-SU056 is understood to also include reference to co-crystal, ester, solvate, hydrate, isomer (including optical isomers, racemates, or other mixtures thereof), tautomer, isotope, polymorph, or a pharmaceutically acceptable prodrug of the enantiomer in question.

Provided is a pharmaceutical composition comprising:
a) a pharmaceutically effective amount of(S)-9-(3-fluorophenyl)-5-(2-hydroxyethyl)-6,9-dihydro-[1,3]dioxolo[4,5-g]furo[3,4-b]quinolin-8(5H)-one ((S)-SU056), or a pharmaceutically acceptable salt thereof, in an enantiomeric excess to (R)-9-(3-fluorophenyl)-5-(2-hydroxyethyl)-6,9-dihydro-[1,3]dioxolo[4,5-g]furo[3,4-b]quinolin-8(5H)-one ((R)-SU056), or a pharmaceutically acceptable salt thereof; and
b) a pharmaceutically acceptable carrier or excipient.

The compositions herein may be used in methods of medical treatment. It is understood that, for each method of treatment or inhibition described herein, there is an embodiment wherein the use of the term “a composition” refers to a composition of (S)-SU056), or a pharmaceutically acceptable salt (or other forms listed above) thereof in an enantiomeric excess to (R)-SU056), or a pharmaceutically acceptable salt (or other forms listed above) thereof. For each method there is also an embodiment in which the use of “a composition” refers to a pharmaceutical composition of (S)-SU056) or the other (S)-SU056 forms listed above, in an enantiomeric excess to (R)-SU056) or the other (R)-SU056 forms listed above and a pharmaceutically acceptable excipient or carrier.

Also provided is a method of inhibiting YB1 protein activity in a subject experiencing a cancer expressing YB1 protein, the method comprising administering to the subject in need thereof a pharmaceutically effective amount of a composition comprising(S)-9-(3-fluorophenyl)-5-(2-hydroxyethyl)-6,9-dihydro-[1,3]dioxolo[4,5-g]furo[3,4-b]quinolin-8(5H)-one ((S)-SU056), or a pharmaceutically acceptable salt thereof, in an enantiomeric excess to (R)-9-(3-fluorophenyl)-5-(2-hydroxyethyl)-6,9-dihydro-[1,3]dioxolo[4,5-g]furo[3,4-b]quinolin-8(5H)-one ((R)-SU056), or a pharmaceutically acceptable salt thereof.

Further provided is a method of sensitizing cancer cells expressing the YB1 protein in a subject to treatment with an anticancer agent, the method comprising administering to the subject in need thereof a pharmaceutically effective amount of a composition comprising(S)-9-(3-fluorophenyl)-5-(2-hydroxyethyl)-6,9-dihydro-[1,3]dioxolo[4,5-g]furo[3,4-b]quinolin-8(5H)-one ((S)-SU056), or a pharmaceutically acceptable salt thereof, in an enantiomeric excess to (R)-9-(3-fluorophenyl)-5-(2-hydroxyethyl)-6,9-dihydro-[1,3]dioxolo[4,5-g]furo[3,4-b]quinolin-8(5H)-one ((R)-SU056), or a pharmaceutically acceptable salt thereof.

Additionally provided is a method of sensitizing cancer cells expressing the YB1 protein in a subject to treatment with radiation, the method comprising administering to the subject in need thereof a pharmaceutically effective amount of a composition comprising(S)-9-(3-fluorophenyl)-5-(2-hydroxyethyl)-6,9-dihydro-[1,3]dioxolo[4,5-g]furo[3,4-b]quinolin-8(5H)-one ((S)-SU056), or a pharmaceutically acceptable salt thereof, in an enantiomeric excess to (R)-9-(3-fluorophenyl)-5-(2-hydroxyethyl)-6,9-dihydro-[1,3]dioxolo[4,5-g]furo[3,4-b]quinolin-8(5H)-one ((R)-SU056), or a pharmaceutically acceptable salt thereof.

The methods herein of treatment and sensitization in a subject of a cancer expressing YB1 protein includes those for cancers selected from the group of gynecological cancers (including ovarian, endometrial, fallopian tube, and cervical cancers), breast cancers, lung cancers, prostate cancer, colorectal cancer, bladder cancer, melanoma, liver cancer, multiple myeloma, soft tissue sarcoma, osteosarcoma, Ewing's sarcoma, glioblastoma, acute myeloid leukemia, Chronic myelogenous leukemia, acute lymphoblastic leukemia, chronic lymphocytic leukemia, lymphoma, kidney cancer, renal cell carcinoma, osteosarcoma, pancreatic cancer, head and neck cancer, nasopharyngeal carcinoma, and gastric cancer.

It is understood that the pharmaceutically effective amount of compositions described herein may be administered in a regimen concurrently with an additional anticancer agent or radiation. In other embodiments, the pharmaceutically effective amount of the composition described herein may be administered to a subject in need thereof in a dose or regimen prior to subsequent administration of a designated cancer agent or agents and/or radiation therapy.

In some embodiments, the composition described herein may be administered for an initial period of time, such as from 1 to 7 days, followed in sequence by administration to the subject in need thereof of a designated cancer agent or agents and/or radiation therapy.

In further embodiments, the composition described herein and one or more designated cancer agent or agents and/or radiation therapy may be administered to the subject in need thereof in repeating sequential periods of time, such as from 1 to 14 days each, with or without a refractory period involving neither treatment in between each pair of administrations.

In other embodiments, the composition described herein may be administered for an initial period of time, such as from 1 to 7 days, followed by a second period of co-administration to the subject in need thereof of both composition described herein and a pharmaceutically effective amount of a designated cancer agent or agents and/or radiation therapy.

In different embodiments, the cancer cells expressing YB1 (YBX1) protein in a subject sensitized to or by the treatments described herein are selected from the group of a gynecological cancer (including ovarian, endometrial, fallopian tube, and cervical cancers), leukemias, lymphomas, kidney cancer, bladder cancer, pancreatic cancer, head and neck cancer, breast cancers (including triple negative, ER-negative, ER-positive breast cancers, and progesterone-positive), lung cancers, ovarian cancer, prostate cancer, colorectal cancer, gastric cancer, and neuronal cancer (including gliomas).

### Gynecological Cancers

Provided is a method of treatment of gynecological cancers expressing YB1 (YBX1) protein in a subject, the method comprising administering to the subject in need thereof a pharmaceutically effective amount of a composition described herein.

Also provided is a method of treatment of gynecological cancers expressing YB1 (YBX1) protein in a subject, the method comprising administering to the subject in need thereof a pharmaceutically effective amount of a composition described herein, and a pharmaceutically effective amount of an mTOR inhibitor, or a pharmaceutically acceptable salt thereof. In some embodiments, the mTOR inhibitor is selected from the group of sirolimus, everolimus, deforolimus, and temsirolimus.

Also provided is a method of enhancing the effect of an anticancer agent in a subject experiencing a gynecological cancer expressing YB1 (YBX1) protein, the method comprising administering to the subject in need thereof a pharmaceutically effective amount of a composition described herein. In some embodiments, the treatment with a composition described herein sensitizes gynecological cancer cells expressing YB1 (YBX1) protein in the subject to the treatment of the anticancer agent.

In some embodiments, the anticancer agent used to treat the gynecological cancer is an inhibitor or antagonist of phosphoinositide 3-kinase (PI3K)/protein kinase B (Akt).

In some embodiments, the gynecological cancer expressing YB1 (YBX1) protein to be treated is ovarian cancer. In other embodiments, the gynecological cancer to be treated is endometrial cancer. In still other embodiments, the gynecological cancer to be treated is cervical cancer.

As such, also provided is a method of treatment of ovarian cancer expressing YB1 (YBX1) protein in a subject, the method comprising administering to the subject in need thereof:
a) a pharmaceutically effective amount of a composition described herein; and
b) a pharmaceutically effective amount of cisplatin, or a pharmaceutically acceptable salt thereof.

In some embodiments, the taxane compound used in the method of treatment of the gynecological cancers expressing YB1 (YBX1) protein discussed herein is selected from the group of paclitaxel, docetaxel, and cabazitaxel.

For each of the methods of sensitizing cancer cells expressing YB1 (YBX1) protein in a subject to treatment with an anticancer agent and/or radiation or inhibiting YB1 protein activity in a subject experiencing a cancer, there is a corresponding method with the initial step of detecting the presence or absence of expressed YB1 (YBX1) protein in sample cells of the cancer and, when YB1 (YBX1) protein is determined to be present in the sample cells, treating the subject experiencing the cancer in question as described for each method with a pharmaceutically effective amount of a composition described herein, and any other agent or agents indicated by the particular method.

As a non-limiting example, also provided is a method of treatment of ovarian cancer expressing YB1 (YBX1) protein in a subject, the method comprising the steps of:
a) determining the presence or absence of expressed YB1 protein in an ovarian cancer tumor sample collected from the subject in need thereof; and
b) when expressed YB1 protein is determined to be present in the ovarian cancer tumor sample, administering to the subject in need thereof:
   i) a pharmaceutically effective amount of a composition described herein; and
   ii) a pharmaceutically effective amount of a taxane compound, or a pharmaceutically acceptable salt thereof.

As such, also provided is a method of treatment of fallopian tube cancer (fallopian tube carcinoma) expressing YB1 (YBX1) protein in a subject, the method comprising administering to the subject in need thereof:
a) a pharmaceutically effective amount of a composition described herein; and
b) a pharmaceutically effective amount of a taxane compound, or a pharmaceutically acceptable salt thereof.

As such, also provided is a method of treatment of fallopian tube cancer (fallopian tube carcinoma) expressing YB1 (YBX1) protein in a subject, the method comprising administering to the subject in need thereof:
a) a pharmaceutically effective amount of a composition described herein;
b) a pharmaceutically effective amount of a taxane compound, or a pharmaceutically acceptable salt thereof; and
c) a pharmaceutically effective amount of carboplatin, or a pharmaceutically acceptable salt thereof.

In some embodiments, the taxane compound in the methods of treating fallopian tube cancer is selected from the group of paclitaxel, albumin-bound paclitaxel, docetaxel, and cabazitaxel.

### Prostate Cancer

Also provided is a method of treatment of prostate cancer expressing YB1 (YBX1) protein in a subject, the method comprising administering to the subject in need thereof a pharmaceutically effective amount of a composition described herein.

Further provided is a method of inhibiting prostate cancer metastasis expressing YB1 (YBX1) protein in a subject, the method comprising administering to the subject in need thereof a pharmaceutically effective amount of a composition described herein.

Additionally provided is a method of sensitizing prostate cancer expressing YB1 (YBX1) protein in a subject to treatment with an anticancer agent, the method comprising administering to the subject in need thereof a pharmaceutically effective amount of a composition described herein.

In some embodiments, the administration of a composition described herein sensitizes the prostate cancer expressing YB1 (YBX1) protein in the subject to treatment with a taxane anticancer agent. In some embodiments, the taxane anticancer agent is selected from the group of paclitaxel, docetaxel, and cabazitaxel, or a pharmaceutically acceptable salt thereof.

As such, also provided is a method of treatment of prostate cancer expressing YB1 (YBX1) protein in a subject, the method comprising administering to the subject in need thereof:
a) a pharmaceutically effective amount of a composition described herein; and
b) a pharmaceutically effective amount of a taxane compound selected from the group of paclitaxel, docetaxel, and cabazitaxel, or a pharmaceutically acceptable salt thereof.

In some embodiments, the administration of a composition described herein sensitizes the prostate cancer expressing YB1 (YBX1) protein in the subject to treatment with an androgen receptor inhibitor anticancer agent. In some embodiments the androgen receptor inhibitor is selected from the group of apalutamide, enzalutamide, darolutamide, and abiraterone acetate.

As such, also provided is a method of treatment of prostate cancer expressing YB1 (YBX1) protein in a subject, the method comprising administering to the subject in need thereof:
a) a pharmaceutically effective amount of a composition described herein; and
b) a pharmaceutically effective amount of an androgen receptor inhibitor compound selected from the group of apalutamide, enzalutamide, darolutamide, and abiraterone acetate, or a pharmaceutically acceptable salt thereof.

In some embodiments, the apalutamide is administered to the subject in need thereof at a daily dosage of from about 100 mg to about 300 mg. In some embodiments, the apalutamide is administered to the subject in need thereof at a daily dosage of from about 200 mg to about 300 mg. In some embodiments, the apalutamide is administered at a dosage of about 240 mg per day.

In some embodiments, the anticancer agent is a luteinizing hormone-releasing hormone (LHRH) agonist. In some embodiments, the LHRH agonist is selected from the group of leuprolide/leuprorelin, goserelin, triptorelin, buserelin, and histrelin.

As such, also provided is a method of treatment of prostate cancer expressing YB1 (YBX1) protein in a subject, the method comprising administering to the subject in need thereof:
a) a pharmaceutically effective amount of a composition described herein; and
b) a pharmaceutically effective amount of a luteinizing hormone-releasing hormone (LHRH) agonist compound selected from the group of leuprolide/leuprorelin, goserelin, triptorelin, buserelin, and histrelin, or a pharmaceutically acceptable salt thereof.

In other embodiments, the anticancer agent is a luteinizing hormone-releasing hormone (LHRH) antagonist. In some embodiments, the LHRH agonist is degarelix. As such, also provided is a method of treatment of prostate cancer in a subject, the method comprising administering to the subject in need thereof:
a) a pharmaceutically effective amount of a composition described herein; and
b) a pharmaceutically effective amount of degarelix, or a pharmaceutically acceptable salt thereof.

In other embodiments, the anticancer agent is an anti-androgen agent. In some embodiments, the anti-androgen agent is selected from the group of flutamide, bicalutamide, and nilutamide.

As such, also provided is a method of treatment of prostate cancer expressing YB1 (YBX1) protein in a subject, the method comprising administering to the subject in need thereof:
a) a pharmaceutically effective amount of a composition described herein; and
b) a pharmaceutically effective amount of an anti-androgen compound selected from the group of flutamide, bicalutamide, and nilutamide, or a pharmaceutically acceptable salt thereof.

In some embodiments concerning the methods herein for treating prostate cancer and/or inhibiting prostate cancer metastasis, the prostate cancer in question is an androgen-independent prostate cancer. In other embodiments, the prostate cancer in question is castration-sensitive prostate cancer. In other embodiments, the prostate cancer in question is metastatic castration-sensitive prostate cancer. In additional embodiments, the prostate cancer expressing YB1 (YBX1) protein to be treated is non-metastatic castration-resistant prostate cancer. In other embodiments, the prostate cancer is hormone-refractory prostate cancer (HRPC).

### Melanoma

Also provided is a method of treatment of melanoma expressing YB1 (YBX1) protein in a subject, the method comprising administering to the subject in need thereof a pharmaceutically effective amount of a composition described herein.

Further provided is a method of inhibiting melanoma metastasis expressing YB1 (YBX1) protein in a subject, the method comprising administering to the subject in need thereof a pharmaceutically effective amount of a composition described herein.

Additionally provided is a method of sensitizing melanoma cells expressing YB1 (YBX1) protein in a subject to treatment with an anticancer agent, the method comprising administering to the subject in need thereof a pharmaceutically effective amount of a composition described herein.

As such, also provided is a method of treatment of melanoma expressing YB1 (YBX1) protein in a subject, the method comprising administering to the subject in need thereof:
a) a pharmaceutically effective amount of a composition described herein; and
b) a pharmaceutically effective amount of a PD-1 inhibitor agent selected from the group of pembrolizumab and nivolumab, or a pharmaceutically acceptable salt thereof.

Further provided is a method of treatment of melanoma expressing YB1 (YBX1) protein in a subject, the method comprising administering to the subject in need thereof:
a) a pharmaceutically effective amount of a composition described herein; and
b) a pharmaceutically effective amount of atezolizumab, or a pharmaceutically acceptable salt thereof.

Further provided is a method of treatment of melanoma expressing YB1 (YBX1) protein in a subject, the method comprising administering to the subject in need thereof:
a) a pharmaceutically effective amount of a composition described herein;
b) a pharmaceutically effective amount of atezolizumab, or a pharmaceutically acceptable salt thereof; and
c) a pharmaceutically effective amount of a third agent selected from the group of cobimetinib and vemurafenib, or a pharmaceutically acceptable salt thereof.

Also provided is a method of treatment of melanoma expressing YB1 (YBX1) protein in a subject, the method comprising administering to the subject in need thereof:
a) a pharmaceutically effective amount of a composition described herein; and
b) a pharmaceutically effective amount of a CTLA-4 inhibitor (such as ipilimumab).

As such, also provided is a method of treatment of melanoma expressing YB1 (YBX1) protein in a subject, the method comprising administering to the subject in need thereof:
a) a pharmaceutically effective amount of a composition described herein; and
b) a pharmaceutically effective amount of interleukin-2 (IL-2).

### Cisplatin Resistance

YB-1 expression or overexpression has also been associated with resistance to cisplatin treatments in some cancers, including breast, bladder, and ovarian cancers.

In some embodiments, the breast cancer to be treated is refractory to endocrine therapeutics, such as selective estrogen receptor modulators (SERMs), including tamoxifen and toremifene. In some embodiments, the breast cancer is refractory to selective estrogen receptor degrader (SERDs), such as fulvestrant and elacestrant. In other embodiments, the breast cancer to be treated is refractory to aromatase inhibitors, such as letrozole, anastrozole, exemestane, and testolactone.

Provided herein is a method of sensitizing a cancer expressing YB1 (YBX1) protein in a subject to treatment with cisplatin, the method comprising administering to the subject in need thereof a pharmaceutically effective amount of a composition described herein.

Provided herein is a method of sensitizing a cancer expressing YB1 (YBX1) protein in a subject to treatment with a taxane compound, the method comprising administering to the subject in need thereof a pharmaceutically effective amount of a composition described herein.

### Breast Cancer

Also provided is a method of treatment of breast cancer expressing YB1 (YBX1) protein in a subject, the method comprising administering to the subject in need thereof a pharmaceutically effective amount of a composition described herein.

Further provided is a method of inhibiting breast cancer metastasis expressing YB1 (YBX1) protein in a subject, the method comprising administering to the subject in need thereof a pharmaceutically effective amount of a composition described herein.

Additionally provided is a method of sensitizing breast cancer cells expressing YB1 (YBX1) protein in a subject to treatment with an anticancer agent, the method comprising administering to the subject in need thereof a pharmaceutically effective amount of a composition described herein. In some embodiments, the method sensitizes breast cancer cells expressing YB1 (YBX1) protein in the subject in need thereof to treatment with one or more agents selected from the group of anthracyclines (such as doxorubicin, pegylated liposomal doxorubicin, and epirubicin), taxane compounds (such as paclitaxel, albumin-bound paclitaxel, docetaxel, and cabazitaxel), 5-fluorouracil, capecitabine, cyclophosphamide, vinarelbine, gemcitabine, ixabepilone, eribulin, and platinum agents (such as carboplatin and cisplatin). In other embodiments, the method sensitizes breast cancer cells expressing YB1 (YBX1) to treatment with radiation therapy.

An embodiment provides a method of treatment of breast cancer expressing YB1 (YBX1) protein in a subject, the method comprising administering to the subject in need thereof:
a) a pharmaceutically effective amount of a composition described herein; and
b) a pharmaceutically effective amount of one or more anticancer agents selected from the group of doxorubicin, pegylated liposomal doxorubicin, epirubicin, paclitaxel, docetaxel, 5-fluorouracil, capecitabine, cyclophosphamide, and carboplatin, or a pharmaceutically acceptable salt thereof.

Another embodiment provides a method of treatment of breast cancer expressing YB1 (YBX1) protein in a subject, the method comprising administering to the subject in need thereof:
a) a pharmaceutically effective amount of a composition described herein; and
b) a pharmaceutically effective amount of one or more anticancer agents selected from the group of paclitaxel, albumin-bound paclitaxel, docetaxel, doxorubicin, pegylated liposomal doxorubicin, epirubicin, cisplatin, carboplatin, vinorelbine, capecitabine, gemcitabine, ixabepilone, and eribulin, or a pharmaceutically acceptable salt thereof.

### Colorectal Cancer

Also provided is a method of treatment of colorectal cancer expressing YB1 (YBX1) protein in a subject, the method comprising administering to the subject in need thereof a pharmaceutically effective amount of a composition described herein.

Further provided is a method of inhibiting colorectal cancer metastasis expressing YB1 (YBX1) protein in a subject, the method comprising administering to the subject in need thereof a pharmaceutically effective amount of a composition described herein.

Additionally provided is a method of sensitizing colorectal cancer cells expressing YB1 (YBX1) protein in a subject to treatment with an anticancer agent, the method comprising administering to the subject in need thereof a pharmaceutically effective amount of a composition described herein.

As such, also provided is a method of treatment of colorectal cancer expressing YB1 (YBX1) protein in a subject, the method comprising administering to the subject in need thereof:
a) a pharmaceutically effective amount of a composition described herein; and
b) a pharmaceutically effective amount of an anti-cancer agent selected from the group of 5-fluorouracil, capecitabine, irinotecan, oxaliplatin, and trifluridine and tipiracil, or a pharmaceutically acceptable salt thereof.

For each of the methods of treatment herein utilizing a combination of trifluridine and tipiracil (trifluidine+tipiracil), the combination of agents may include in different embodiments the use of individual dose of a) from about 10 mg to about 20 mg of trifluridine and from about 3 mg to about 10 mg of tipiracil; b) from about 12 mg to about 18 mg of trifluridine and from about 4 mg to about 8 mg of tipiracil; c) from about 14 mg to about 16 mg of trifluridine and from about 5 mg to about 7 mg of tipiracil; d) from about 10 mg to about 30 mg of trifluridine and from about 5 mg to about 10 mg of tipiracil; and e) from about 15 mg to about 25 mg of trifluridine and from about 6 mg to about 9 mg of tipiracil. In some embodiments, the daily dose of the two agents comprises from about 60 mg to about 80 mg of trifluridine and from about 20 mg to about 40 mg of tipiracil. In some embodiments, the daily dose of the two agents comprises from about 65 mg to about 75 mg of trifluridine and from about 25 mg to about 35 mg of tipiracil. In some embodiments, the daily dose is given in a twice daily administration of from about 30 mg to about 40 mg of trifluridine and from about 12 mg to about 16 mg trifluridine.

### Bladder Cancer

Also provided is a method of treatment of bladder cancer expressing YB1 (YBX1) protein in a subject, the method comprising administering to the subject in need thereof a pharmaceutically effective amount of a composition described herein.

Further provided is a method of inhibiting bladder cancer metastasis expressing YB1 (YBX1) protein in a subject, the method comprising administering to the subject in need thereof a pharmaceutically effective amount of a composition described herein.

Additionally provided is a method of sensitizing bladder cancer cells expressing YB1 (YBX1) protein in a subject to treatment with an anticancer agent, the method comprising administering to the subject in need thereof a pharmaceutically effective amount of a composition described herein.

Provided is a method of treatment of bladder cancer expressing YB1 (YBX1) protein in a subject, the method comprising administering to the subject in need thereof:
d) a pharmaceutically effective amount of a composition described herein;
e) a pharmaceutically effective amount of an anticancer agent selected from the group of cisplatin, cisplatin plus 5-fluorouracil, and mitomycin with 5-fluorouracil, or a pharmaceutically acceptable salt thereof;
f) a therapeutically effective dose of radiation.

Also provided is a method of treatment of bladder cancer expressing YB1 (YBX1) protein in a subject, the method comprising administering to the subject in need thereof:
a) a pharmaceutically effective amount of a composition described herein; and
b) a pharmaceutically effective amount of an anticancer agent selected from the group of
   i) gemcitabine and cisplatin;
   ii) Dose-dense methotrexate, vinblastine, doxorubicin (Adriamycin), and cisplatin (DDMVAC);
   iii) Cisplatin, methotrexate, and vinblastine (CMV); and
   iv) Gemcitabine and paclitaxel

Also provided is a method of treatment of bladder cancer expressing YB1 (YBX1) protein in a subject, the method comprising administering to the subject in need thereof:
a) a pharmaceutically effective amount of a composition described herein;
b) a pharmaceutically effective amount of an anticancer agent selected from the group of docetaxel, paclitaxel, doxorubicin, methotrexate, ifosfamide, and pemetrexed, or a pharmaceutically acceptable salt thereof.

### Liver Cancer

Also provided is a method of treatment of liver cancer expressing YB1 (YBX1) protein in a subject, the method comprising administering to the subject in need thereof a pharmaceutically effective amount of a composition described herein.

Further provided is a method of inhibiting liver cancer metastasis expressing YB1 (YBX1) protein in a subject, the method comprising administering to the subject in need thereof a pharmaceutically effective amount of a composition described herein.

Additionally provided is a method of sensitizing liver cancer cells expressing YB1 (YBX1) protein in a subject to treatment with an anticancer agent, the method comprising administering to the subject in need thereof a pharmaceutically effective amount of a composition described herein.

As such, also provided is a method of treatment of liver cancer expressing YB1 (YBX1) protein in a subject, the method comprising administering to the subject in need thereof:
a) a pharmaceutically effective amount of a composition described herein; and
b) a pharmaceutically effective amount of an anti-cancer agent selected from the group of gemcitabine, oxaliplatin, cisplatin, doxorubicin, 5-fluorouracil, capecitabine, and mitoxantrone, or a pharmaceutically acceptable salt thereof.

### Lung Cancer

Also provided is a method of treatment of small cell lung cancer expressing YB1 (YBX1) protein in a subject, the method comprising administering to the subject in need thereof a pharmaceutically effective amount of a composition described herein.

Further provided is a method of inhibiting small cell lung cancer metastasis expressing YB1 (YBX1) protein in a subject, the method comprising administering to the subject in need thereof a pharmaceutically effective amount of a composition described herein.

Additionally provided is a method of sensitizing small cell lung cancer cells expressing YB1 (YBX1) protein in a subject to treatment with an anticancer agent, the method comprising administering to the subject in need thereof a pharmaceutically effective amount of a composition described herein.

As such, also provided is a method of treatment of small cell lung cancer expressing YB1 (YBX1) protein in a subject, the method comprising administering to the subject in need thereof:
a) a pharmaceutically effective amount of a composition described herein; and
b) a pharmaceutically effective amount of an anti-cancer agent selected from the group of cisplatin and etoposide, carboplatin and etoposide, and irinotecan, and carboplatin and irinotecan, or a pharmaceutically acceptable salt thereof.

Also provided is a method of treatment of non-small cell lung cancer expressing YB1 (YBX1) protein in a subject, the method comprising administering to the subject in need thereof a pharmaceutically effective amount of a composition described herein.

Further provided is a method of inhibiting non-small cell lung cancer metastasis expressing YB1 (YBX1) protein in a subject, the method comprising administering to the subject in need thereof a pharmaceutically effective amount of a composition described herein.

Additionally provided is a method of sensitizing non-small cell lung cancer cells expressing YB1 (YBX1) protein in a subject to treatment with an anticancer agent, the method comprising administering to the subject in need thereof a pharmaceutically effective amount of a composition described herein.

As such, also provided is a method of treatment of non-small cell lung cancer expressing YB1 (YBX1) protein in a subject, the method comprising administering to the subject in need thereof:
a) a pharmaceutically effective amount of a composition described herein; and
b) a pharmaceutically effective amount of one or more anti-cancer agents selected from the group of cisplatin, carboplatin, paclitaxel, albumin-bound paclitaxel, docetaxel, gemcitabine, vinarelbine, etoposide, and pemetrexed, or a pharmaceutically acceptable salt thereof.

### Multiple Myeloma

Also provided is a method of treatment of multiple myeloma expressing YB1 (YBX1) protein in a subject, the method comprising administering to the subject in need thereof a pharmaceutically effective amount of a composition described herein.

Further provided is a method of inhibiting multiple myeloma metastasis expressing YB1 (YBX1) protein in a subject, the method comprising administering to the subject in need thereof a pharmaceutically effective amount of a composition described herein.

Additionally provided is a method of sensitizing multiple myeloma cells expressing YB1 (YBX1) protein in a subject to treatment with an anticancer agent, the method comprising administering to the subject in need thereof a pharmaceutically effective amount of a composition described herein.

As such, also provided is a method of treatment of multiple myeloma expressing YB1 (YBX1) protein in a subject, the method comprising administering to the subject in need thereof:
a) a pharmaceutically effective amount of a composition described herein; and
b) a pharmaceutically effective amount of an anti-cancer agent selected from the group of Melphalan, vincristine, cyclophosphamide, etoposide, doxorubicin, liposomal doxorubicin, and bendamustine, or a pharmaceutically acceptable salt thereof.

### Soft Tissue Sarcomas

Also provided herein are methods of treatment of soft tissue sarcomas expressing YB1 (YBX1) protein, including angiosarcoma, dermatofibrosarcoma protuberans, epitheloid sarcoma, gastrointestinal stromal tumor (GIST), Kaposi's sarcoma, Leiomyosarcoma, liposarcoma, malignant peripheral nerve sheath tumors, myxofibrosarcoma, rhabdomyosarcoma, solitary fibrous tumors, synovial sarcoma, and undifferentiated pleomorphic sarcoma.

Also provided is a method of treatment of soft tissue sarcomas expressing YB1 (YBX1) protein, such as fibrosarcoma expressing YB1 (YBX1) protein, in a subject, the method comprising administering to the subject in need thereof a pharmaceutically effective amount of a composition described herein.

Further provided is a method of inhibiting soft tissue sarcomas expressing YB1 (YBX1) protein, such as fibrosarcoma expressing YB1 (YBX1) protein, in a subject, the method comprising administering to the subject in need thereof a pharmaceutically effective amount of a composition described herein.

Additionally provided is a method of sensitizing soft tissue sarcoma cells expressing YB1 (YBX1) protein, such as fibrosarcoma cells expressing YB1 (YBX1) protein, in a subject to treatment with an anticancer agent, the method comprising administering to the subject in need thereof a pharmaceutically effective amount of a composition described herein.

As such, also provided is a method of treatment of soft tissue sarcomas expressing YB1 (YBX1) protein, such as fibrosarcoma expressing YB1 (YBX1) protein, in a subject, the method comprising administering to the subject in need thereof:
a) a pharmaceutically effective amount of a composition described herein; and
b) a pharmaceutically effective amount of one or more anti-cancer agents selected from the group of ifosfamide, doxorubicin, dacarbazine (DTIC), epirubicin, temozolomide, docetaxel, gemcitabine, vinorelbine, trabectedin, and eribulin, or a pharmaceutically acceptable salt thereof.

In some embodiments, when the anti-cancer agent ifosfamide is used, the drug mesna is also given to protect the bladder from the toxic effects of ifosfamide. In other embodiments, the anti-cancer agent is a combination of mesna, Adriamycin [doxorubicin], ifosfamide, and dacarbazine, sometimes referred to by the acronym MAID. In other embodiments, the anti-cancer agent is a combination of Adriamycin [doxorubicin], ifosfamide, and mesna, sometimes referred to by the acronym AIM. In some embodiments in the methods of treating soft tissue sarcomas herein, the anti-cancer agent or agents are administered to the subject in need thereof using isolated limb perfusion.

Osteosarcomas: Also provided is a method of treatment of osteosarcoma expressing YB1 (YBX1) protein in a subject, the method comprising administering to the subject in need thereof a pharmaceutically effective amount of a composition described herein.

Further provided is a method of inhibiting osteosarcoma expressing YB1 (YBX1) protein in a subject, the method comprising administering to the subject in need thereof a pharmaceutically effective amount of a composition described herein.

Additionally provided is a method of sensitizing osteosarcoma cells expressing YB1 (YBX1) protein in a subject to treatment with an anticancer agent, the method comprising administering to the subject in need thereof a pharmaceutically effective amount of a composition described herein.

As such, also provided is a method of treatment of osteosarcoma expressing YB1 (YBX1) protein in a subject, the method comprising administering to the subject in need thereof:
a) a pharmaceutically effective amount of a composition described herein; and
b) a pharmaceutically effective amount of one or more anti-cancer agents selected from the group of methotrexate, doxorubicin, cisplatin, carboplatin, Ifosfamide, cyclophosphamide, etoposide, and gemcitabine, or a pharmaceutically acceptable salt thereof.

In some embodiments in the method of treating osteosarcoma expressing YB1 (YBX1) protein above, the anti-cancer agent is a combination of High-dose methotrexate, doxorubicin, and cisplatin (the MAP regimen). In other embodiments, a combination of doxorubicin and cisplatin are administered. In other embodiments, a combination of ifosfamide and etoposide are used. In still other embodiments, a combination is administered of ifosfamide and epirubicin with either cisplatin or carboplatin.

### Ewing's Sarcoma

Also provided is a method of treatment of Ewing's sarcoma expressing YB1 (YBX1) protein in a subject, the method comprising administering to the subject in need thereof a pharmaceutically effective amount of a composition described herein.

Further provided is a method of inhibiting Ewing's sarcoma expressing YB1 (YBX1) protein in a subject, the method comprising administering to the subject in need thereof a pharmaceutically effective amount of a composition described herein.

Additionally provided is a method of sensitizing Ewing's sarcoma cells expressing YB1 (YBX1) protein in a subject to treatment with an anticancer agent, the method comprising administering to the subject in need thereof a composition described herein.

As such, also provided is a method of treatment of Ewing's sarcoma expressing YB1 (YBX1) protein in a subject, the method comprising administering to the subject in need thereof:
a) a pharmaceutically effective amount of a composition described herein; and
b) a pharmaceutically effective amount of one or more anti-cancer agents selected from the group of cyclophosphamide, doxorubicin, etoposide, Ifosfamide, and vincristine, or a pharmaceutically acceptable salt thereof.

In some embodiments in the method of treating Ewing's sarcoma expressing YB1 (YBX1) protein above, the anti-cancer agent is a combination of vincristine, doxorubicin, and cyclophosphamide, alternating with ifosfamide and etoposide, the regimen referred to as VDC/IE.

### Gastric Cancers

Also provided is a method of treatment of gastric (stomach) cancer expressing YB1 (YBX1) protein in a subject, the method comprising administering to the subject in need thereof a pharmaceutically effective amount of a composition described herein.

Further provided is a method of inhibiting gastric cancer expressing YB1 (YBX1) protein in a subject, the method comprising administering to the subject in need thereof a pharmaceutically effective amount of a composition described herein.

Additionally provided is a method of sensitizing gastric cancer cells expressing YB1 (YBX1) protein in a subject to treatment with an anticancer agent, the method comprising administering to the subject in need thereof a pharmaceutically effective amount of a composition described herein.

As such, also provided is a method of treatment of gastric cancer expressing YB1 (YBX1) protein in a subject, the method comprising administering to the subject in need thereof:
a) a pharmaceutically effective amount of a composition described herein; and
b) a pharmaceutically effective amount of one or more anti-cancer agents selected from the group of 5-fluorouracil, capecitabine, carboplatin, cisplatin, docetaxel, epirubicin, irinotecan, oxaliplatin, paclitaxel, and trifluridine+tipiracil (LONSURF®) or a pharmaceutically acceptable salt thereof.

In some embodiments in the method of treating gastric cancer expressing YB1 (YBX1) protein above, the anti-cancer agent is a combination of epirubicin, cisplatin, and 5-fluorouracil, sometimes referred to by the acronym ECF. In other embodiments, the combination of docetaxel or paclitaxel with either 5-FU or capecitabine, sometimes combined with radiation. In another embodiment cisplatin is administered with either 5-FU or capecitabine, sometimes combined with radiation. In further embodiments paclitaxel and carboplatin are administered, sometimes combined with radiation. In other embodiments the combination of docetaxel, cisplatin, and 5-fluorouracil (DCF) are administered. In others, irinotecan is administered along with cisplatin, 5-fluorouracil, or capecitabine. In still others, oxaliplatin is administered with 5-fluorouracil or capecitabine. In still others, trifluridine+tipiracil (LONSURF®) is given.

### Glioblastoma

Also provided is a method of treatment of glioblastoma multiforme (GBM or glioblastoma) expressing YB1 (YBX1) protein in a subject, the method comprising administering to the subject in need thereof a pharmaceutically effective amount of a composition described herein.

Further provided is a method of inhibiting glioblastoma expressing YB1 (YBX1) protein in a subject, the method comprising administering to the subject in need thereof a pharmaceutically effective amount of a composition described herein.

Additionally provided is a method of sensitizing glioblastoma cells expressing YB1 (YBX1) protein in a subject to treatment with an anticancer agent, the method comprising administering to the subject in need thereof a pharmaceutically effective amount of a composition described herein.

As such, also provided is a method of treatment of glioblastoma expressing YB1 (YBX1) protein in a subject, the method comprising administering to the subject in need thereof:
a) a pharmaceutically effective amount of a composition described herein; and
b) a pharmaceutically effective amount of one or more anti-cancer agents selected from the group of temozolomide, bevacizumab, lomustine, carmustine, fluzoparil, pembrolizumab, nivolumab, ipilimumab, anlotinib, glasdegib, and bavituximab, or a pharmaceutically acceptable salt thereof.

In some embodiments, the glioblastoma in the methods above is a pediatric glioblastoma expressing YB1 (YBX1) protein. In some embodiments the glioblastoma is a primary glioblastoma expressing YB1 (YBX1) protein. In others, it is a secondary glioblastoma expressing YB1 (YBX1) protein.

### Head and Neck Cancer

Reference to “head and neck cancer” herein refers to any of the cancers of the oral cavity, throat (pharynx, including the nasopharynx, oropharynx, and hypopharynx), larynx, paranasal sinuses, nasal cavity, and salivary glands. The head and neck cancers include Hypopharyngeal cancer, laryngeal cancer, lip and oral cavity cancer, metastatic squamous neck cancer, nasopharyngeal cancer, oropharyngeal cancer, paranasal sinus and nasal cavity cancer, and salivary gland cancer. It is understood that, for each of the methods of treatment of head and neck cancer described herein, disclosed also is the corresponding method for each of the head and neck cancers listed in this paragraph.

Also provided is a method of treatment of head and neck cancer expressing YB1 (YBX1) protein in a subject, the method comprising administering to the subject in need thereof a pharmaceutically effective amount of a composition described herein.

Further provided is a method of inhibiting head and neck cancer metastasis expressing YB1 (YBX1) protein in a subject, the method comprising administering to the subject in need thereof a pharmaceutically effective amount of a composition described herein.

Additionally provided is a method of sensitizing head and neck cancer expressing YB1 (YBX1) protein in a subject to treatment with an anticancer agent, the method comprising administering to the subject in need thereof a pharmaceutically effective amount of a composition described herein.

In some embodiments concerning the treatment of head and neck cancer expressing YB1 (YBX1), the anticancer agent used to treat the subject is radiation therapy. In some embodiments, the radiation utilized is external-beam radiation therapy. In some embodiments, the treatment comprises administering a pharmaceutically effective amount of one or more EGFR inhibitors to the subject in need thereof. Other embodiments concern respectively administering a pharmaceutically effective amount of larotrectinib (Vitrakvi) and/or larotrectinib to the subject in need thereof.

Other methods of treating head and neck cancer comprise the use of immunotherapy, such as the administration a pharmaceutically effective amount of pembrolizumab and/or nivolumab to the subject in need thereof.

Provided is a method of treatment of head and neck cancer expressing YB1 (YBX1) protein in a subject, the method comprising administering to the subject in need thereof:
a) a pharmaceutically effective amount of a composition described herein; and
b) a pharmaceutically effective amount of an anticancer agent selected from the group of paclitaxel, docetaxel, cisplatin, carboplatin, 5-fluorouracil, methotrexate, and capecitabine, or a pharmaceutically acceptable salt thereof.

As such, also provided is a method of treatment of head and neck cancer expressing YB1 (YBX1) protein in a subject, the method comprising administering to the subject in need thereof:
a) a pharmaceutically effective amount of a composition described herein; and
b) a pharmaceutically effective amount of a taxane compound selected from the group of paclitaxel and docetaxel, or a pharmaceutically acceptable salt thereof.

Also provided is a method of treatment of head and neck cancer expressing YB1 (YBX1) protein in a subject, the method comprising administering to the subject in need thereof:
a) a pharmaceutically effective amount of a composition described herein; and
b) a pharmaceutically effective amount of a cisplatin, or a pharmaceutically acceptable salt thereof.

In some embodiments, the cisplatin is administered to the subject in need thereof at a dose of from about 20 mg/m² to about 100 mg/m² delivered every 3 weeks×3.

Also provided is a method of treatment of head and neck cancer expressing YB1 (YBX1) protein in a subject, the method comprising administering to the subject in need thereof:
a) a pharmaceutically effective amount of a composition described herein; and
b) a pharmaceutically effective amount of a carboplatin, or a pharmaceutically acceptable salt thereof; and
c) a pharmaceutically effective amount of a drug selected from the group of 5-fluorouracil (5FU) and cetuximab.

Also provided is a method of treatment of head and neck cancer expressing YB1 (YBX1) protein in a subject, the method comprising administering to the subject in need thereof:
a) a pharmaceutically effective amount of a composition described herein; and
b) a pharmaceutically effective amount of cisplatin, or a pharmaceutically acceptable salt thereof; and
c) a pharmaceutically effective amount of a drug selected from the group of 5-fluorouracil (5FU) and cetuximab.

Also provided is a method of treatment of head and neck cancer expressing YB1 (YBX1) protein in a subject, the method comprising administering to the subject in need thereof:
a) a pharmaceutically effective amount of a composition described herein; and
b) a pharmaceutically effective amount of cisplatin, or a pharmaceutically acceptable salt thereof; and
c) a pharmaceutically effective amount of paclitaxel, or a pharmaceutically acceptable salt thereof.

Also provided is a method of treatment of head and neck cancer expressing YB1 (YBX1) protein in a subject, the method comprising administering to the subject in need thereof:
a) a pharmaceutically effective amount of a composition described herein; and
b) a pharmaceutically effective amount of carboplatin, or a pharmaceutically acceptable salt thereof; and
c) a pharmaceutically effective amount of paclitaxel, or a pharmaceutically acceptable salt thereof.

Also provided is a method of treatment of head and neck cancer expressing YB1 (YBX1) protein in a subject, the method comprising administering to the subject in need thereof:
a) a pharmaceutically effective amount of a composition described herein; and
b) a pharmaceutically effective amount of hydroxyurea, or a pharmaceutically acceptable salt thereof; and
c) a pharmaceutically effective amount of a drug selected from the group of 5-fluorouracil (5FU) and cetuximab.

Further provided is a method of treatment of nasopharyngeal cancer expressing YB1 (YBX1) protein in a subject, the method comprising administering to the subject in need thereof:
a) a pharmaceutically effective amount of a composition described herein; and
b) a pharmaceutically effective amount of hydroxyurea, or a pharmaceutically acceptable salt thereof; and
c) a pharmaceutically effective amount of a drug selected from the group of carboplatin, doxorubicin, epirubicin, paclitaxel, docetaxel, gemcitabine, bleomycin, and methotrexate.

### Pancreatic Cancer

Provided is a method of treatment of pancreatic cancer expressing YB1 (YBX1) protein in a subject, the method comprising administering to the subject in need thereof a pharmaceutically effective amount of a composition described herein.

Further provided is a method of inhibiting pancreatic cancer metastasis expressing YB1 (YBX1) protein in a subject, the method comprising administering to the subject in need thereof a pharmaceutically effective amount of a composition described herein.

Additionally provided is a method of sensitizing pancreatic cancer expressing YB1 (YBX1) protein in a subject to treatment with an anticancer agent, the method comprising administering to the subject in need thereof a pharmaceutically effective amount of a composition described herein.

Provided is a method of treatment of pancreatic cancer expressing YB1 (YBX1) protein in a subject, the method comprising administering to the subject in need thereof:
a) a pharmaceutically effective amount of a composition described herein; and
b) a pharmaceutically effective amount of one or more anticancer agents selected from the group of gemcitabine, 5-fluorouracil, oxaliplatin, paclitaxel, albumin-bound paclitaxel, docetaxel, capecitabine, cisplatin, and irinotecan, or a pharmaceutically acceptable salt thereof.

### Neuronal Cancers

The compositions described herein may also be used to treat and/or sensitize to treatment neuronal cancers (brain and spinal cord cancers), including medulloblastoma, glioblastoma multiforme (GBM), astrocytomas (anaplastic astrocytomas and pilocytic astrocytomas), ependymomas, and oligodendrogliomas. It is understood for each of the following methods for treating a neuronal cancer or sensitizing a neuronal cancer to treatment described herein, included separate methods of each type for each of the neuronal cancers listed in this paragraph.

Provided is a method of treatment of neuronal cancer expressing YB1 (YBX1) protein in a subject, the method comprising administering to the subject in need thereof a pharmaceutically effective amount of a composition described herein.

Further provided is a method of inhibiting neuronal cancer metastasis expressing YB1 (YBX1) protein in a subject, the method comprising administering to the subject in need thereof a pharmaceutically effective amount of a composition described herein.

Additionally provided is a method of sensitizing neuronal cancer expressing YB1 (YBX1) protein in a subject to treatment with an anticancer agent, the method comprising administering to the subject in need thereof a pharmaceutically effective amount of a composition described herein.

Provided is a method of treatment of neuronal cancer expressing YB1 (YBX1) protein in a subject, the method comprising administering to the subject in need thereof:
a) a pharmaceutically effective amount of a composition described herein; and
b) a pharmaceutically effective amount of one or more anticancer agents selected from the group of carboplatin, carmustine (BCNU), cisplatin, irinotecan, cyclophosphamide, etoposide, lomustine, methotrexate, procarbazine, temozolomide, and vincristine, or a pharmaceutically acceptable salt thereof.

In some embodiments, the pharmaceutically effective amount of carmustine administered to the subject in need thereof is in the form of a carmustine wafer or implant, such as that in the GLIADEL® Wafer (carmustine implant) product available from Arbor Pharmaceuticals, LLC.

### Leukemias

Methods of the present invention also include those for the treatment of leukemias, wherein the leukemia cells in question express YB-1 protein, including acute myeloid leukemia (AML), Chronic myelogenous leukemia (CML), acute lymphoblastic (or lymphocytic) leukemia (ALL), and chronic lymphocytic leukemia (CLL). It is understood that for each method described herein for a treatment for leukemia or of sensitizing a leukemia to a treatment, separate corresponding methods are understood for each of the referenced leukemias (AML, CML, ALL, and CLL).

Additionally provided is a method of sensitizing leukemia cells expressing YB1 (YBX1) protein in a subject to treatment with an anticancer agent, the method comprising administering to the subject in need thereof a pharmaceutically effective amount of a composition described herein. In some embodiments, the anticancer agent used to treat the subject is radiation therapy.

Also provided is a method of treatment of acute myeloid leukemia expressing YB1 (YBX1) protein in a subject, the method comprising administering to the subject in need thereof:
a) a pharmaceutically effective amount of a composition described herein; and
b) a pharmaceutically effective amount of an anthracycline drug selected from the group of daunorubicin and idarubicin, or a pharmaceutically acceptable salt thereof; and
c) a pharmaceutically effective amount of cytarabine, or a pharmaceutically acceptable salt thereof

Also provided is a method of treatment of acute myeloid leukemia expressing YB1 (YBX1) protein in a subject, the method comprising administering to the subject in need thereof:
a) a pharmaceutically effective amount of a composition described herein; and
b) a pharmaceutically effective amount of one or more anticancer agents selected from the group of cladribine (2-CdA), fludarabine, mitoxantrone, etoposide, 6-thioguanine, hydroxyurea, prednisone, dexamethasone, methotrexate, 6-mercaptopurine, azacitidine, and decitabine, or a pharmaceutically acceptable salt thereof.

Also provided is a method of treatment of chronic myeloid leukemia expressing YB1 (YBX1) protein in a subject, the method comprising administering to the subject in need thereof:
a) a pharmaceutically effective amount of a composition described herein; and
b) a pharmaceutically effective amount of one or more anticancer agents selected from the group of hydroxyurea, cytarabine (Ara-C), busulfan, cyclophosphamide (CYTOXAN®), and vincristine (ONCOVIN®), or a pharmaceutically acceptable salt thereof

Also provided is a method of treatment of chronic myeloid leukemia expressing YB1 (YBX1) protein in a subject, the method comprising administering to the subject in need thereof:
a) a pharmaceutically effective amount of a composition described herein; and
b) a pharmaceutically effective amount of one or more tyrosine kinase inhibitor anticancer agents selected from the group of imatinib (GLEEVEC®), dasatinib (SPRYCEL®), nilotinib (TASIGNA®), bosutinib (BOSULIF®), ponatinib (ICLUSIG®), and asciminib (SCEMBLIX®), or a pharmaceutically acceptable salt thereof.

Also provided is a method of treatment of chronic myeloid leukemia expressing YB1 (YBX1) protein in a subject, the method comprising administering to the subject in need thereof:
a) a pharmaceutically effective amount of a composition described herein; and
b) a pharmaceutically effective amount of interferon-alpha, or a pharmaceutically acceptable salt thereof.

Also provided is a method of treatment of acute lymphoblastic leukemia expressing YB1 (YBX1) protein in a subject, the method comprising administering to the subject in need thereof:
a) a pharmaceutically effective amount of a composition described herein; and
b) a pharmaceutically effective amount of one or more anticancer agents selected from the group of vincristine, dexamethasone, imatinib, prednisone, doxorubicin and daunorubicin, or a pharmaceutically acceptable salt thereof.

Also provided is a method of treatment of acute lymphoblastic leukemia expressing YB1 (YBX1) protein in a subject, the method comprising administering to the subject in need thereof:
a) a pharmaceutically effective amount of a composition described herein; and
b) a pharmaceutically effective amount of one or more anticancer agents selected from the group of methotrexate, 6-mercaptopurine, vincristine, prednisone, and imatinib, or a pharmaceutically acceptable salt thereof.

Also provided is a method of treatment of acute lymphoblastic leukemia expressing YB1 (YBX1) protein in a subject, the method comprising administering to the subject in need thereof:
a) a pharmaceutically effective amount of a composition described herein; and
b) a pharmaceutically effective amount of one or more anticancer agents selected from the group of vincristine, dexamethasone, prednisone, doxorubicin, and daunorubicin, or a pharmaceutically acceptable salt thereof.

Also provided is a method of treatment of acute lymphoblastic leukemia expressing YB1 (YBX1) protein in a subject, the method comprising administering to the subject in need thereof:
a) a pharmaceutically effective amount of a composition described herein; and
b) a pharmaceutically effective amount of one or more anticancer agents selected from the group of methotrexate, 6-mercaptopurine (6-MP), vincristine, prednisone, and imatinib, or a pharmaceutically acceptable salt thereof.

Also provided is a method of treatment of chronic lymphocytic leukemia expressing YB1 (YBX1) protein in a subject, the method comprising administering to the subject in need thereof:
a) a pharmaceutically effective amount of a composition described herein; and
b) a pharmaceutically effective amount of one or more anticancer agents selected from the group of ibrutinib, acalabrutinib, idelalisib, and duvelisib, or a pharmaceutically acceptable salt thereof.

Also provided is a method of treatment of chronic lymphocytic leukemia expressing YB1 (YBX1) protein in a subject, the method comprising administering to the subject in need thereof:
a) a pharmaceutically effective amount of a composition described herein; and
b) a pharmaceutically effective amount of venetoclax, or a pharmaceutically acceptable salt thereof.

Also provided is a method of treatment of chronic lymphocytic leukemia expressing YB1 (YBX1) protein in a subject, the method comprising administering to the subject in need thereof:
a) a pharmaceutically effective amount of a composition described herein; and
c) a pharmaceutically effective amount of one or more anticancer agents selected from the group of rituximab, ofatumumab, and obinutuzumab, or a pharmaceutically acceptable salt thereof.

### Definitions

The terms “YB1” and “YBX1” refer to Y box binding protein 1, also known as Y-box transcription factor or nuclease-sensitive element-binding protein 1, a protein that in humans is encoded by the YBX1 gene.

In some embodiments, a “subject in need thereof” concerning a method of treatment herein is a patient from whom a tumor sample, such as from a tumor biopsy, is taken and the presence of expressed YBX1 protein is identified in the sampled material, such as through immunohistochemical or Western Blotting techniques known in the art.

The terms “effective amount,” “therapeutically effective amount,” or “pharmaceutically effective amount” refer to an amount that is sufficient to effect treatment, as defined below, when administered to a subject (e.g., a mammal, such as a human) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, an “effective amount,” “therapeutically effective amount,” or a “pharmaceutically effective amount” of a composition described herein, is an amount sufficient to modulate YBX1 expression or activity, and thereby treat a subject (e.g., a human) suffering an indication, or to ameliorate or alleviate the existing symptoms of the indication. For example, a therapeutically or pharmaceutically effective amount may be an amount sufficient to decrease a symptom of a disease or condition responsive to inhibition of YBX1 activity.

In some embodiments, an “effective amount” is an amount of a subject compound that, when administered to an individual in one or more doses, in monotherapy or in combination therapy, is effective to inhibit YB-1 by about 20% (20% inhibition), at least about 30% (30% inhibition), at least about 40% (40% inhibition), at least about 50% (50% inhibition), at least about 60% (60% inhibition), at least about 70% (70% inhibition), at least about 80% (80% inhibition), or at least about 90% (90% inhibition), compared to the YB-1 activity in the individual in the absence of treatment with the compound, or alternatively, compared to the YB-1 activity in the individual before or after treatment with the compound.

In some embodiments, an “effective amount” is an amount of a subject compound that, when administered to an individual in one or more doses, in monotherapy or in combination therapy, is effective to decrease tumor burden in the subject by about 20%, at least about 30%, at least about 40%, at least about 50%, at least about 60%, at least about 70%, at least about 80%, or at least about 90%, compared to tumor burden in the individual in the absence of treatment with the compound, or alternatively, compared to the tumor burden in the subject before or after treatment with the compound. As used herein the term “tumor burden” refers to the total mass of tumor tissue carried by a subject with cancer. In some embodiments, an “effective amount” is an amount of a subject compound that, when administered to an individual in one or more doses, in monotherapy or in combination therapy, is effective to reduce the dose of radiotherapy required to observe tumor shrinkage in the subject by about 20%, at least about 30%, at least about 40%, at least about 50%, at least about 60%, at least about 70%, at least about 80%, or at least about 90%, compared to the dose of radiotherapy required to observe tumor shrinkage in the individual in the absence of treatment with the compound. In some embodiments, an “effective amount” of a compound is an amount that, when administered in one or more doses to an individual having cancer, is effective to achieve a 1.5-log, a 2-log, a 2.5-log, a 3-log, a 3.5-log, a 4-log, a 4.5-log, or a 5-log reduction in tumor size.

In some embodiments, the(S)-9-(3-fluorophenyl)-5-(2-hydroxyethyl)-6,9-dihydro-[1,3]dioxolo[4,5-g]furo[3,4-b]quinolin-8(5H)-one, or a pharmaceutically acceptable salt thereof, may be administered at a daily dose of from about 0.2 mg/kg to about 10 mg/kg. In other embodiments, it may be administered at a daily dose of from about 0.2 mg/kg to about 5 mg/kg.

In some embodiments, an effective amount of(S)-9-(3-fluorophenyl)-5-(2-hydroxyethyl)-6,9-dihydro-[1,3]dioxolo[4,5-g]furo[3,4-b]quinolin-8(5H)-one, or a pharmaceutically acceptable salt thereof, is an amount that ranges from about 50 ng/kg body weight to about 50 pg/kg body weight (e.g., from about 50 ng/kg body weight to about 40 pg/kg body weight, from about 30 ng/kg body weight to about 20 pg/kg body weight, from about 50 ng/kg body weight to about 10 pg/kg body weight, from about 50 ng/kg body weight to about 1 pg/kg body weight, from about 50 ng/kg body weight to about 800 ng/kg body weight, from about 50 ng/kg body weight to about 700 ng/kg body weight, from about 50 ng/kg body weight to about 600 ng/kg body weight, from about 50 ng/kg body weight to about 500 ng/kg body weight, from about 50 ng/kg body weight to about 400 ng/kg body weight, from about 60 ng/kg body weight to about 400 ng/kg body weight, from about 70 ng/kg body weight to about 300 ng/kg body weight, from about 60 ng/kg body weight to about 100 ng/kg body weight, from about 65 ng/kg body weight to about 85 ng/kg body weight, from about 70 ng/kg body weight to about 90 ng/kg body weight, from about 200 ng/kg body weight to about 900 ng/kg body weight, from about 200 ng/kg body weight to about 800 ng/kg body weight, from about 200 ng/kg body weight to about 700 ng/kg body weight, from about 200 ng/kg body weight to about 600 ng/kg body weight, from about 200 ng/kg body weight to about 500 ng/kg body weight, from about 200 ng/kg body weight to about 400 ng/kg body weight, or from about 200 ng/kg body weight to about 300 ng/kg body weight).

In some embodiments, an effective amount of(S)-9-(3-fluorophenyl)-5-(2-hydroxyethyl)-6,9-dihydro-[1,3]dioxolo[4,5-g]furo[3,4-b]quinolin-8(5H)-one, or a pharmaceutically acceptable salt thereof, is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 pg, from about 1 pg to about 10 pg, from about 10 pg to about 50 pg, from about 50 mg to about 150 gg, from about 150 gg to about 250 gg, from about 250 gg to about 500 gg, from about 500 gg to about 750 gg, from about 750 gg to about 1 g, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg. In other embodiments, the daily dose or daily amount is from 1 mg to 1,000 mg. In other embodiments, the daily dose or daily amount is from 10 mg to 1,000 mg. In further embodiments, the daily dose or daily amount is from 10 mg to 750 mg. In additional embodiments, the daily dose or daily amount is from 10 mg to 500 mg. In additional embodiments, the daily dose or daily amount is from 100 mg to 500 mg.

In some embodiments, a single dose of(S)-9-(3-fluorophenyl)-5-(2-hydroxyethyl)-6,9-dihydro-[1,3]dioxolo[4,5-g]furo[3,4-b]quinolin-8(5H)-one, or a pharmaceutically acceptable salt thereof, is administered. In other embodiments, multiple doses are administered. Where multiple doses are administered over a period of time, the compound can be administered twice daily (qid), daily (qd), every other day (qod), every third day, three times per week (tiw), or twice per week (biw) over a period of time. For example, a compound is administered qid, qd, qod, tiw, or biw over a period of from one day to about 2 years or more. For example, (S)-9-(3-fluorophenyl)-5-(2-hydroxyethyl)-6,9-dihydro-[1,3]dioxolo[4,5-g]furo[3,4-b]quinolin-8(5H)-one, or a pharmaceutically acceptable salt thereof, is administered at any of the aforementioned frequencies for one week, two weeks, one month, two months, six months, one year, or two years, or more, depending on various factors.

Administration of an effective amount of a(S)-9-(3-fluorophenyl)-5-(2-hydroxyethyl)-6,9-dihydro-[1,3]dioxolo[4,5-g]furo[3,4-b]quinolin-8(5H)-one, or a pharmaceutically acceptable salt thereof, to an individual with cancer can result in one or more of: 1) a reduction in tumor burden; 2) a reduction in the dose of radiotherapy required to effect tumor shrinkage (e.g. resulting from sensitization to radiotherapy); 3) a reduction in the spread of a cancer from one cell to another cell in an individual; 4) a reduction of morbidity or mortality in clinical outcomes; 5) shortening the total length of treatment when combined with other anti-cancer agents (e.g. resulting from sensitization to other anti-cancer agents); and 6) an improvement in an indicator of disease response (e.g., a reduction in one or more symptoms of cancer). Any of a variety of methods can be used to determine whether a treatment method is effective. For example, a biological sample obtained from an individual who has been treated with a subject method can be assayed.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and I, D and L, or (+) and (−) are employed to designate the sign of rotation of plane-polarized light by the compound, with S, (−), or 1 meaning that the compound is levorotatory while a compound prefixed with R, (+), or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

“Substantially free” as used in reference to enantiomers herein refers to a composition comprising (S)-(−)-SU056 in greater amounts by weight to (R)-(+)-SU056. In some embodiments the composition comprises at least 60% by weight (S)-(−)-SU056, or a pharmaceutically acceptable salt thereof, and not more than 40% by weight (R)-(+)-SU056, or a pharmaceutically acceptable salt thereof. In some embodiments the composition comprises at least 70% (S)-(−)-SU056, or a pharmaceutically acceptable salt thereof, and not more than 30% (R)-(+)-SU056, or a pharmaceutically acceptable salt thereof. In other embodiments the composition comprises at least 80% (S)-(−)-SU056, or a pharmaceutically acceptable salt thereof, and not more than 20% (R)-(+)-SU056, or a pharmaceutically acceptable salt thereof. In additional embodiments the composition comprises at least 90% (S)-(−)-SU056, or a pharmaceutically acceptable salt thereof, and not more than 10% (R)-(+)-SU056, or a pharmaceutically acceptable salt thereof. In some embodiments the composition comprises at least 95% (S)-(−)-SU056, or a pharmaceutically acceptable salt thereof, and not more than 5% (R)-(+)-SU056, or a pharmaceutically acceptable salt thereof. In different embodiments the composition comprises at least 98% (S)-(−)-SU056, or a pharmaceutically acceptable salt thereof, and not more than 2% (R)-(+)-SU056, or a pharmaceutically acceptable salt thereof. In further embodiments the composition comprises at least 99% (S)-(−)-SU056, or a pharmaceutically acceptable salt thereof, and not more than 1% (R)-(+)-SU056, or a pharmaceutically acceptable salt thereof. In some embodiments the composition comprises at least 99.9% (S)-(−)-SU056, or a pharmaceutically acceptable salt thereof, and not more than 0.1% (R)-(+)-SU056, or a pharmaceutically acceptable salt thereof. All percentages given for the enantiomers above are weight percentages.

The relative presence or concentration of an enantiomer may also be characterized as an “enantiomeric excess (ee or EE)” regarding the degree to which one enantiomer is present in greater amounts than its counterpart. For instance, a racemic mixture has an enantiomeric excess of 0%, whereas a pure enantiomer has an enantiomeric excess of 100%. A sample with 80% of one enantiomer and 20% of the other has an enantiomeric excess of 60% (80% minus 20%). In separate embodiments, the enantiomeric excess of(S)-(−)-SU056 in relation to (R)-(+)-SU056 may be, respectively, 10%, 20%, 30%, 40%, 50%, 60%, 70%, 80%, 90%, 95%, 96%, 97%, 98%, 99%, 99.5%, and 99.9%. In other embodiments, the composition comprises 100% (S)-(−)-SU056, as the presence of (R)-(+)-SU056 is too low to be detected by conventional means.

“Subject” refers to an animal, such as a mammal, that has been or will be the object of treatment, observation or experiment. The methods described herein may be useful in both human therapy and veterinary applications. In some embodiments, the subject is a mammal; in some embodiments the subject is human; and in some embodiments the subject is chosen from cats and dogs. “Subject in need thereof” or “human in need thereof” refers to a subject, such as a human, who may have or is suspected to have diseases or conditions that would benefit from certain treatment; for example treatment with a composition described herein, or a pharmaceutically acceptable salt or co-crystal thereof, as described herein. This includes a subject who may be determined to be at risk of or susceptible to such diseases or conditions, such that treatment would prevent the disease or condition from developing.

“Treatment” or “treating” is an approach for obtaining beneficial or desired results including clinical results. Beneficial or desired clinical results may include one or more of the following: (i) inhibiting the disease or condition (e.g., decreasing one or more symptoms resulting from the disease or condition, and/or diminishing the extent of the disease or condition); (ii) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (iii) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

The terms “inhibiting” or “inhibition” indicates a decrease, such as a significant decrease, in the baseline activity of a biological activity or process. “Inhibition of YB-1 activity” refers to a decrease in YB-1 activity as a direct or indirect response to the presence of a composition described herein, relative to the activity of YB-1 in the absence of such compound or a pharmaceutically acceptable salt or co-crystal thereof. The decrease in activity may be due to the direct interaction of the compound with YB-1, or due to the interaction of the compound(s) described herein with one or more other factors that in turn affect YB-1 activity. For example, the presence of the compound(s) may decrease YB-1 activity by directly binding to the YB-1, by causing (directly or indirectly) another factor to decrease YB-1 activity, or by (directly or indirectly) decreasing the amount of YB-1 present in the cell or organism. In some embodiments, the inhibition of YB-1 activity may be compared in the same subject prior to treatment, or other subjects not receiving the treatment. The term “inhibitor” is understood to refer to a compound or agent that, upon administration to a human in need thereof at a pharmaceutically or therapeutically effective dose, provides the inhibition activity desired.

The term “pharmaceutical composition” refers to a composition containing a pharmaceutically effective amount of one or more of the isotopic compounds described herein, or a pharmaceutically acceptable salt thereof, formulated with a pharmaceutically acceptable carrier, which can also include other additives, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein. Conventional procedures and ingredients for the selection and preparation of suitable formulations are described, for example, in Remington: The Science and Practice of Pharmacy, 21^{st} Ed., Gennaro, Ed., Lippencott Williams & Wilkins (2005) and in The United States Pharmacopeia: The National Formulary (USP 36 NF31), published in 2013.

As used herein, “pharmaceutically acceptable excipient” is a pharmaceutically acceptable vehicle that includes, without limitation, any and all carriers, solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable carrier” refers to any ingredient in a pharmaceutical composition other than the disclosed pharmaceutically active or therapeutic compounds, or a pharmaceutically acceptable salt thereof (e.g., a carrier capable of suspending or dissolving the active isotopic compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt” includes, for example, salts with inorganic acids and salts with an organic acid. Examples of salts may include hydrochloride, phosphate, diphosphate, hydrobromide, sulfate, sulfinate, nitrate, malate, maleate, fumarate, tartrate, succinate, citrate, acetate, lactate, methanesulfonate (mesylate), benzenesuflonate (besylate), p-toluenesulfonate (tosylate), 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

The term “crystal forms” and related terms herein refer to the various crystalline modifications of a given substance, including, but not limited to, polymorphs, solvates, hydrates, co-crystals, and other molecular complexes, as well as salts, solvates of salts, hydrates of salts, other molecular complexes of salts, and polymorphs thereof. Crystal forms of a substance can be obtained by a number of methods, as known in the art. Such methods include, but are not limited to, melt recrystallization, melt cooling, solvent recrystallization, recrystallization in confined spaces such as, e.g., in nanopores or capillaries, recrystallization on surfaces or templates, such as, e.g., on polymers, recrystallization in the presence of additives, such as, e.g., co-crystal counter-molecules, desolvation, dehydration, rapid evaporation, rapid cooling, slow cooling, vapor diffusion, sublimation, grinding and solvent-drop grinding.

The term “refractory” used herein in regard to a cancer refers to a cancer that does not respond to one or more treatments. In some embodiments, the cancer does not respond to one or more chemotherapeutic agents. In other embodiments, the cancer does not respond to radiation therapy. In other embodiments, the refractory cancer does not respond to one or more chemotherapeutic agents and radiation therapy. A refractory cancer may be resistant at the beginning of such treatments or may become resistant over the course of one or more treatments. In cases of chemotherapy, refractory cancers may also be referred to as “chemotherapy resistant cancers” or “chemo-resistant cancers.” A “platinum resistant cancer” is one in which responds at first to treatment with drugs that contain the metal platinum, such as cisplatin, carboplatin, oxaliplatin, nedaplatin, lobaplatin, triplatin tetranitrate, and picoplatin, but returns within a certain period. For example, ovarian cancer that comes back within 6 months after treatment is considered platinum resistant. For each of the methods of treatment herein, there is a further embodiment wherein the cancer in question is a platinum-resistant cancer.

“Androgen-independent prostate cancer” or “hormone-refractory prostate cancer (AIPC)” is prostate cancer that progresses after primary androgen-ablation therapy, either from orchiectomy or a gonadotropin-releasing hormone (LHRH) agonist, followed by addition and subsequent withdrawal of an antiandrogen. In some embodiments hormone-refractory prostate cancer is defined as 2-3 consecutive rises in prostate-specific antigen (PSA) levels obtained at intervals of greater than 2 weeks and/or documented disease progression based on findings from CT scan and/or bone scan, bone pain, or obstructive voiding symptoms. In some embodiments, the PSA level does not rise at diagnosis or throughout the entire course of the disease. In some embodiments, the prostate cancer is an advanced prostate cancer. In some embodiments, the prostate cancer or metastatic prostate cancer is resistant to treatments with hormone-blocking therapies, such as abiraterone (ZYTIGA®), enzalutamide (XTANDI®), bicalutamide (CASODEX®), flutamide (DROGENIL®), or cyproterone acetate (CYPROSTAT®).

Cancer cells that are resistant to radiation treatment, whether intrinsically or acquired over time, are referred to as “radiation resistant cancers” or “radioresistant cancers.” The terms are intended to describe cancer cells that are less responsive to radiation treatments than non-resistant cancer cells.

All ranges disclosed and/or claimed herein are inclusive of the recited endpoint and independently combinable. For example, the ranges of “from 2 to 10” and “2-10” are inclusive of the endpoints, 2 and 10, and all the intermediate values between in context of the units considered. For instance, reference to “Claims 2-10” or “C₂-C₁₀ alkyl” includes units 2, 3, 4, 5, 6, 7, 8, 9, and 10, as claims and atoms are numbered in sequential numbers without fractions or decimal points, unless described in the context of an average number. The context of “pH of from 5-9” or “a temperature of from 5° C. to 9° C.”, on the other hand, includes whole numbers 5, 6, 7, 8, and 9, as well as all fractional or decimal units in between, such as 6.5 and 8.24.

By “significant” is meant any detectable change that is statistically significant in a standard parametric test of statistical significance such as Student's T-test, where p<0.05.

The modifier “about” used in connection with a quantity is inclusive of the stated value and has the meaning dictated by the context (e.g., includes the degree of error associated with measurement of the particular quantity). In some embodiments the term “about” refers to the amount indicated, plus or minus 10%. In some embodiments the term “about” refers to the amount indicated, plus or minus 5%.

### Procedure for the Synthesis of 9-(3-fluorophenyl)-5-(2-hydroxyethyl)-6,9-dihydro-[1,3]dioxolo[4,5-g]furo[3,4-b]quinolin-8(5H)-one

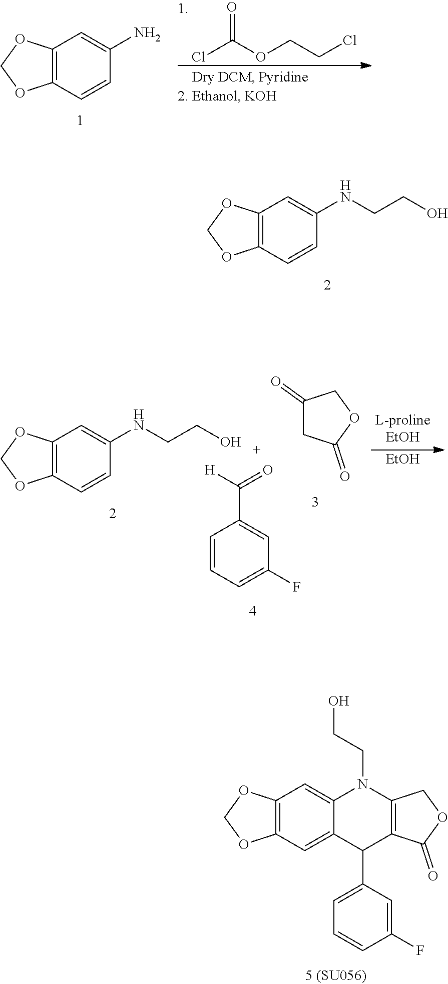

### Experimental Materials and Methods

All the reagents and solvents were obtained at the highest commercial quality from sources such as Sigma-Aldrich, Fisher Scientific, Acros organics, Alfa-Aesar, ChemScene, and Chem-Implex and were used without further purification. The reactions were monitored by TLC using pre-coated silica gel plates (Merck, silica gel 60 F254). Flash chromatography was carried out using a CombiFlash Rf+Lu-men chromatography system (Teledyne ISCO, Lincon, NE, USA). 1H (400 MHz) and 13C (101 MHZ) NMR spectra were recorded either on an Agilent 400-MR NMR or on a Bruker Avance 400 MHz spectrometer, using appropriate deuterated solvents, as needed. Chemical shifts (0) were reported in parts per million (ppm) upfield from tetramethylsilane (TMS) as an internal standard. Coupling constants (J) were reported in hertz (Hz), and s, br.s, d, t and m are designated as singlet, broad singlet, doublet, triplet and multiplet, respectively. LC-MS spectra were recorded on an Agilent 6490 iFunnel Triple Quadrupole Mass Spectrometer from Agilent Technologies Inc. (Santa Clara, CA, USA). An Agilent EclipsePlusC18 reverse phase column, 1.8 μm, 2.1×50 mm was used with solvent A (0.1% formic acid in acetonitrile) and solvent B (0.1% formic acid in water) for LC-MS analysis. The ratio of solvent A and solvent B was 1:9 at the beginning and gradually changed to 9:1 at the end.

### Synthesis of 2-(benzo[d][1,3]dioxol-5-ylamino) ethan-1-ol (2)

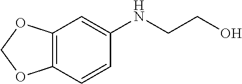 [SMILES: OCCNc1ccc2c(c1)OCO2]

The title compound was prepared according to the previously reported method with several modifications. Under a nitrogen atmosphere, benzo[d][1,3]dioxol-5-amine (30 g, 0.218 mol) was added to dry dichloromethane (500 mL) at room temperature (rt). To this solution, pyridine (22.49 g, 0.284 mol) and 2-chloroethylchloroformate (32.8 g, 0.229 mol) was slowly added with stirring. The mixture was stirred at rt for 3 h, and washed with water (4×100 mL). The organic layer was dried over Na₂SO₄, filtered, and evaporated to dryness using a rotatory evaporator. The crude material was dissolved in ethanol (350 mL), treated with NaOH (35 g, 0.875 mol) and heated at 90° C. overnight. The solvent was evaporated by rotatory evaporator. The solid obtained was dissolved in copious amounts of ethyl acetate (EtOAc), and then washed with water followed by brine solution. The organic phase was dried over Na₂SO₄, filtered and evaporated to dryness to afford crude product, which was purified on a silica gel column using a 0-50% gradient of EtOAc in hexane to obtain the desired product as a pale-brown oil (20 g, 51%).

### Synthesis of 9-(3-fluorophenyl)-5-(2-hydroxyethyl)-6,9-dihydro-[1,3]dioxolo[4,5-g]furo[3,4-b]quinolin-8(5H)-one (5, SU-056)

The title compound was prepared according to the previously reported method, (Kumar, A.; Alegria, A. E., Synthesis of novel functionalized 4-aza-2, 3-didehydropodophyllotoxin derivatives with potential antitumor activity. Journal of heterocyclic chemistry 2010, 47 (6), 1275; Andreoli, M.; Persico, M.; Kumar, A.; Orteca, N.; Kumar, V.; Pepe, A.; Mahalingam, S.; Alegria, A. E.; Petrella, L.; Sevciunaite, L., Identification of the first inhibitor of the GBP1: PIM1 interaction. Implications for the development of a new class of anticancer agents against paclitaxel resistant cancer cells. Journal of medicinal chemistry 2014, 57 (19), 7916-7932; and Resendez, A.; Tailor, D.; Graves, E.; Malhotra, S. V., Radiosensitization of Head and Neck Squamous Cell Carcinoma (HNSCC) by a Podophyllotoxin. ACS medicinal chemistry letters 2019, 10 (9), 1314-1321) with several modifications. Under a nitrogen atmosphere, 2-(benzo[d][1,3]dioxol-5-ylamino) ethan-1-ol 2 (16 g, 0.088 mol), tetronic acid 3 (10.5 g, 0.105 mol), 3-fluorobenzaldehyde 4 (13.5 g, 0.105 mol) and L-proline (1.01 g, 0.008 mmol) were added to dry ethanol (400 mL). The reaction mixture was refluxed overnight. Upon consumption of 2, reaction mixture was evaporated to dryness. The crude product was purified on a silica gel column using a 0-50% gradient of EtOAc in hexane to obtain the desired product. The product was recrystallized from ethanol to afford the desired compound (12.1 g, 37%). 1H NMR (400 MHZ, DMSO-d6) δ 7.29 (td, J=7.9, 6.1 Hz, 1H), 7.13-6.92 (m, 4H), 6.66 (s, 1H), 5.96 (dd, J=24.7, 1.1 Hz, 2H), 5.19-4.91 (m, 4H), 4.04-3.47 (m, 4H). 13C NMR (101 MHz, DMSO-d6) õ 172.64, 163.93, 161.50, 161.15, 150.33, 150.27, 147.54, 143.77, 131.47, 130.68, 130.60, 124.06, 124.04, 118.96, 114.79, 114.58, 113.71, 113.50, 110.50, 101.90, 96.85, 94.81, 66.34, 58.37, 48.63. LC-MS (ESI-QQQ): m/z 370.1 ([C₂₀H₁₆FNO₅+H]+ calcd 370.1). Purity >99.5% (RT 4.35 min).

### Separation of Enantiomers:

The chiral separation of 5, SU-056 was achieved by normal-phase HPLC. Normal-phase chiral HPLC analysis was performed on a Waters preparative 150Q LC system, which is equipped with a 2545 quaternary pump, 2707 automated sample injector, Waters column heater module, and 2998 photodiode array detector. Data were collected and processed by the Empower 3 software. The SU-056 enantiomers were separated on a Lux Cellulose-4 [cellulose tris(4-chloro-3-methylphenylcarbamate), 250×21.2 mm (ID), 5 μm] column.

A 1.2 g amount of racemic compound was dissolved in 6 mL of DMSO and then injected 400 μL per run. An isocratic elution of 50:50 with solvent A (Hexane) and solvent B (Ethanol) was used for enantiomeric separation. The flow rate was set to 10 mL/min with the detection wavelength at 320 nm. Run time was set to 50 min. Column oven temperature was set to 40° C. Fraction collection was set up through Empower 3 software for threshold collection within windows. A pair of collection windows was defined (14.0 to 22.0 and 30.0 to 40.0 min), and any peaks that eluted outside of this window were not collected. Enantiomer 1 eluted between 14.0 and 22.0 min and enantiomer 2 eluted between 30.0 and 40.0 min. The pure fractions were combined and evaporated to dryness to afford the desired enantiomer.

The purity of each enantiomer was checked using an analytical column Lux Cellulose-4, 100×4.6 mm (ID), 5 μm column, eluting with a gradient of 3:7 Hex/EtOH to 1:9 Hex/EtOH for the first 5 min then a gradient of 1:9 Hex/EtOH to 0.5:9.5 Hex/EtOH in the next 10 min. In this gradient, enantiomer-1 and enantiomer-2 elute at 4.07 and 5.37 min, respectively. Purity analysis of each enantiomer showed UV area % purity of >99% and 95.7% for enantiomer-1 and enantiomer-2, respectively, based on HPLC analysis.

It is understood that the SU056 enantiomers described herein may be separated, optionally as salt forms, using techniques known in the art, including crystallization/recrystallization, diastereomers formation, chiral chromatography, and enzyme reactions.

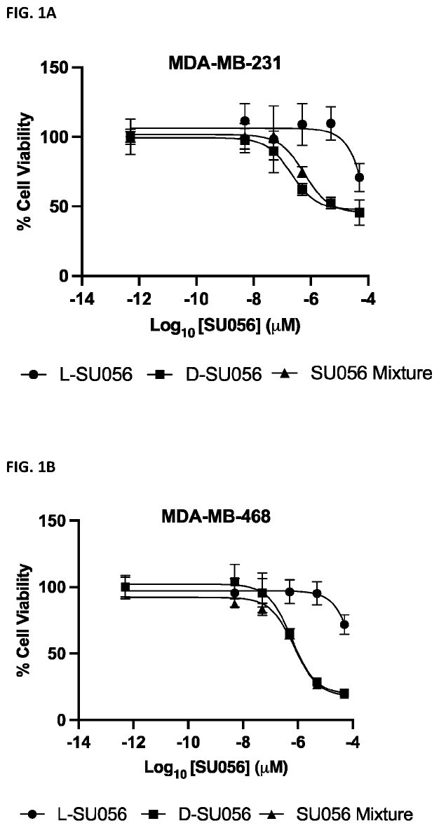

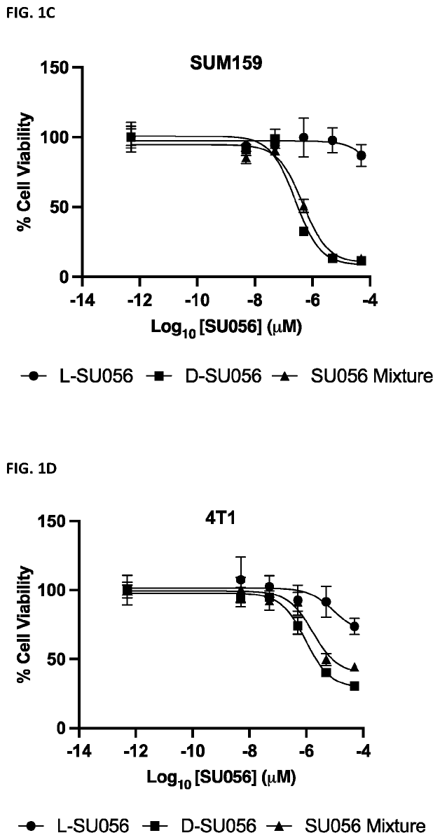

FIGS. 1A, 1B, 1C, and 1D represent the results of cell viability assays using MTT assay in different triple negative breast cancer (MDA-MB-231, MDA-MB-468, SUM159 and 4T1) cells. Cells were plated in 96 well plate and next day, SU056 mixture, Peak 1 of SU056 (D-SU056) and Peak 2 of SU056 (L-SU056) were added to the cells in different log doses (−12.3 to −4.3)) for 48 hr. At the end of 48 hr, cell viability were measured using MTT reagent.

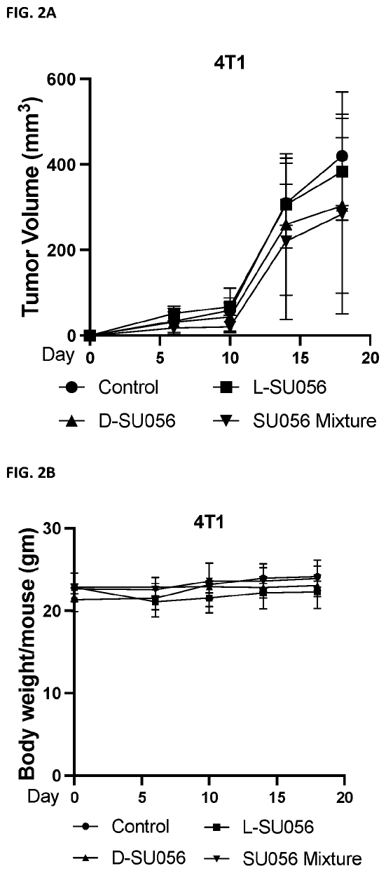

FIGS. 2A and 2B represent the results of a tumor xenograft study of SU056 and its enantiomers in 4T1 tumor xenograft in BALB/c. Mice were subcutaneously injected with 4T1 cells and drug treatment started after three day of implantation. Mice were given either vehicle (40% PEG in saline) or SU056, peak 1 of SU056 and peak 2 of SU056 (50 mg/kg) through oral route using oral gavage. A) Tumor volume (4T1) as a function of time. B) Body weight (4T1) as a function of time.

1. A composition comprising(S)-9-(3-fluorophenyl)-5-(2-hydroxyethyl)-6,9-dihydro-[1,3]dioxolo[4,5-g]furo[3,4-b]quinolin-8(5H)-one, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, in an enantiomeric excess of (R)-9-(3-fluorophenyl)-5-(2-hydroxyethyl)-6,9-dihydro-[1,3]dioxolo[4,5-g]furo[3,4-b]quinolin-8(5H)-one, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

3-15. (canceled)

16. The composition of claim 1, wherein enantiomeric excess is at least 95%.

17. The composition of claim 1, wherein enantiomeric excess is at least 99%.

18. The composition of claim 1, wherein enantiomeric excess is at least 99.9%.

19. A pharmaceutical composition comprising a pharmaceutically effective amount of a composition of claim 1, and a pharmaceutically acceptable carrier or excipient.

20. A method of treatment of a cancer expressing YB1 protein in a subject, the method comprising administering to the subject in need thereof a pharmaceutically effective amount of a composition of claim 1.

21. The method of claim 7, wherein the cancer expressing YB1 protein is selected from the group consisting of ovarian cancer, endometrial cancer, fallopian tube cancer, and cervical cancer, breast cancer, lung cancers, prostate cancer, colorectal cancer, bladder cancer, melanoma, liver cancer, multiple myeloma, soft tissue sarcoma, osteosarcoma, Ewing's sarcoma, glioblastoma, acute myeloid leukemia, chronic myelogenous leukemia, acute lymphoblastic leukemia, chronic lymphocytic leukemia, lymphoma, kidney cancer, renal cell carcinoma, osteosarcoma, pancreatic cancer, head and neck cancer, nasopharyngeal carcinoma, and gastric cancer.

22. The method of claim 8, wherein the cancer expressing YB1 (YBX1) protein in a subject is an ovarian cancer, the method further comprising administering to the subject in need thereof a pharmaceutically effective amount of a taxane compound, or a pharmaceutically acceptable salt thereof.

23. The method of claim 22, wherein the taxane compound is selected from the group consisting of paclitaxel, docetaxel, and cabazitaxel, or a pharmaceutically acceptable salt thereof.

24. The method of claim 22, wherein the taxane compound is paclitaxel, or a pharmaceutically acceptable salt thereof.

25. The method of claim 22, wherein the cancer in question is resistant to treatment with one or more agents from the group consisting of cisplatin, carboplatin, oxaliplatin, nedaplatin, lobaplatin, triplatin tetranitrate, and picoplatin.

26. The method of claim 21, wherein the cancer expressing YB1 (YBX1) protein in a subject is a breast cancer, the method further comprising administering to the subject in need thereof a pharmaceutically effective amount of a taxane compound, or a pharmaceutically acceptable salt thereof.

27. The method of claim 21, wherein the cancer expressing YB1 (YBX1) protein in a subject is a breast cancer, the method further comprising administering to the subject in need thereof a pharmaceutically effective amount of one or more anticancer agents selected from the group consisting of doxorubicin, pegylated liposomal doxorubicin, epirubicin, paclitaxel, docetaxel, 5-fluorouracil, capecitabine, cyclophosphamide, and carboplatin, or a pharmaceutically acceptable salt thereof.

28. The method of claim 21, wherein the cancer expressing YB1 (YBX1) protein in a subject is a breast cancer, the method further comprising administering to the subject in need thereof a pharmaceutically effective amount of one or more anticancer agents selected from the group consisting of paclitaxel, albumin-bound paclitaxel, docetaxel, doxorubicin, pegylated liposomal doxorubicin, epirubicin, cisplatin, carboplatin, vinorelbine, capecitabine, gemcitabine, ixabepilone, and eribulin, or a pharmaceutically acceptable salt thereof.
